(19) United States
(12) Patent Application Publication
(10) Pub. No.: US 2025/0228795 A1
(43) Pub. Date: Jul. 17, 2025

(54) ESTROGENIC COMPOUNDS AND METHODS OF USE

(71) Applicant: laterion, Inc., Oakland, CA (US)

(72) Inventors: Isaac Cohen, Oakland, CA (US); Dale C. Leitman, Oakland, CA (US)

(21) Appl. No.: 19/038,613

(22) Filed: Jan. 27, 2025

Related U.S. Application Data
(63) Continuation of application No. PCT/US2023/028874, filed on Jul. 27, 2023.
(60) Provisional application No. 63/393,635, filed on Jul. 29, 2022.

(51) Int. Cl. A61K 31/12; A61K 31/4402; A61K 31/4406; A61K 31/4409; C07C 49/248; C07C 225/22; C07D 213/50

(52) U.S. Cl. CPC A61K 31/12; A61K 31/4402; A61K 31/4406; A61K 31/4409; C07C 49/248; C07C 225/22; C07D 213/50

(57) ABSTRACT

Estrogenic compounds and compositions, and a method, are presented. The novel estrogenic compounds comprise novel chalcone derivatives and analogs that have little or no measurable estrogen (estradiol, E2) activity but potentiate E2 activity without binding an estrogen receptor (ER). The novel compositions comprise one or more of the novel estrogenic compounds and may optionally comprise one or more excipients. The one or more excipients may be pharmaceutically acceptable. The one or more excipients may comprise at least one compound that does not occur in nature with estrogenic compounds. The method comprises administering a pharmaceutical composition comprising an estrogenic compound described herein to a patient in need of treatment with an estrogenic compound or pharmaceutical composition.

### CROSS-REFERENCE

This application claims the benefit of U.S. Provisional Application No. 63/393,635 filed Jul. 29, 2022, the contents of which are incorporated herein by reference in their entirety.

### FIELD

The present invention is directed to estrogenic compounds, compositions, and methods, including compositions for use in the prevention, treatment, or reduction in severity and/or frequency symptoms of menopause.

### BACKGROUND

Estrogen is a steroid hormone involved in control of multiple developmental and physiological function in both males and females. Estrogen is not just an estrus-inducing sex hormone. Estrogens are pivotal for survival and health in both genders, though their quantity, adaptive response, tissue-specific distribution, and receptor affinity vary with different phases of life. Estrogen is indispensable to glucose homeostasis, immune robustness, bone health, cardiovascular health, fertility, and neural functions.

Estrogens (estradiol (E2) and related steroidal hormones) are biosynthesized mainly in the gonads but also in the adrenal cortex and adipose tissue. Estrogen regulation is mediated by nuclear estrogen receptors (ERs) and the membrane estrogen receptor (GPER1), which are encoded by different genes on different chromosomes, suggesting differential functions. However, estrogen is at the center of almost all human pathologies as well—e.g., infectious, autoimmune, metabolic and degenerative conditions. Both hypo-and hyper-estrogen levels have been linked to chronic and acute diseases. While normal aging results in a significantly lower level of estrogen, leading to tissue degeneration (bone, muscle, neural etc.), and metabolite imbalance (glucose, lipid etc.), the increase in inflammatory agents in day-to-day life enhances the level of estrogen (or estrogen mimic), which is of critical pathophysiological importance. The resultant excess estrogen induces fluctuations in estrogen receptors alpha and beta (ERα and ERβ), harming tissues, leading to autoimmune diseases, and neoplasms.

Estrogen is involved in growth, development, and tissue differentiation from the embryonic phase to death. Puberty gives rise to rapid physiological changes like a growth spurt of bone and muscle mass, and maturation of the gonads and the brain. Estrogen is a crucial signaling contributor to all these processes and especially to sexual differentiation. Hormone fluctuations and disruptions lead to multiple pathologies at this age, not all of which are directly associated with just sexual development and function. There is a world-wide increase in pre-mature puberty which results in increased female cancer risks and reduces girl-specific quality of life.

During menarche estrogen levels fluctuate significantly throughout the menstrual cycle. Early menarche and extended menarche, even at normal levels of cycle fluctuations, result in significant increase in risk of aging diseases such as breast cancer and type 2 diabetes. A majority of women (up to 91%) experience painful menstruation and 14% experience heavy bleeding. There is a world-wide increase in female infertility, which is not just due to increase in age of first pregnancy. Up to 21% of couples suffer infertility, with up to 80% due to female disorders. Despite improved fertility technologies such as in-vitro fertilization (IVF), the success rate of these technologies is very low (approximately 9% for IVF). Up to 46% of women experience spontaneous abortion (miscarriage). There is increasing trend of gestational diabetes, preterm labor, preeclampsia and gestational anemia, all associated with estrogen function.

In women of reproductive age, estrogen is mainly synthesized in the granulosa cells of ovarian follicles. As women enter menopause, the follicles in the ovaries are depleted due to atresia during each menstrual cycle, and the amount of estrogen produced by the ovaries declines. When estrogen levels begin to drop and fluctuate, short-term symptoms such as hot flashes, night sweats, and mood changes frequently arise. The ovaries will eventually stop producing estrogen, and the duration of estrogen deficiency will increase, accelerating the risk of chronic diseases such as osteoporosis, cardiovascular disease, obesity, type 2 diabetes, and urogenital atrophy.

Approximately 70% of all autoimmune diseases are diagnosed in women including rheumatoid arthritis, lupus, Sjogren's syndrome, scleroderma, Hashimoto's thyroiditis, multiple sclerosis among others. As women age there is increased incidence of breast cancer and ovarian cancer. More so, approximately 70% of cases of Alzheimer's disease and early dementia are diagnosed in women.

Estrogen pharmaceutical intervention, e.g. menopausal hormone therapy (MHT), is the most common intervention in all female-related disorders. Natural estrogen and synthetic derivatives are used in these prescriptions. The mechanism of action of these estrogens are as universal agonists, resulting in activation and repression of genes. There are several synthetic steroidal and non-steroidal estrogen receptor antagonists used mostly for the treatment of breast cancer. Despite the pharmacological intention to achieve estrogen receptor antagonism, resulting in gene regulation and pathophysiological outcome, which is unlike the natural hormone, most steroidal and non-steroidal drugs result in a mixed agonist/antagonist outcome, whereby they result in opposite outcome to estrogen in some tissues, while resulting in similar outcome in other tissues. These are the selective estrogen receptor modulators (SERMs), now used for the treatment of breast cancer, osteoporosis and, when combined with estrogen, for menopausal symptoms.

Historically, to oppose the increase in uterine cancer risk associated with menopausal hormone therapy, progestogens are co-prescribed with estrogens. Progestogens exert their pharmacological action via the progesterone receptor.

In recent decades, since the discovery of the ERβ in 1995, attempts have been made to develop receptor subtype agonist and antagonist drugs. None of these drugs are approved by regulators.

Menopausal hormone therapy (MHT) is recommended for only five years to treat menopausal and perimenopausal vasomotor symptoms and vulvovaginal atrophy, as longer term administration of MHT has been shown to increase the risk of estrogen-related adverse outcomes, such as breast cancer, stroke, venous thrombosis (VTE), and Alzheimer's disease. Nonetheless, treatment with MHT, in addition to treating vasomotor symptoms of menopause, also reduces the risk of osteoporosis and type 2 diabetes mellitus (T2DM), cardiovascular disease, obesity, and urogenital atrophy. Thus, there is a need for therapeutic options having one or more advantages of MHT with decreased risk of adverse outcomes, such as increased cancer risk associated with traditional MHT (E2, alone or on combination with a progestogen, or a SERM).

There is a need for additional and novel therapeutic compounds, compositions, and methods for the treatment of symptoms of menopause and perimenopause. The various embodiments disclosed herein address these needs and provide related advantages as well.

### BRIEF SUMMARY OF THE INVENTION

In the current invention we propose a new class of drugs to pharmacologically modulate estrogen receptor (ER) activity. These are the nuclear receptor reprogramming (NRRP) drugs, which are described herein below in the specification and the appended claims and figures. These drugs are neither agonists nor antagonists, nor do they result in a mixed agonist/antagonist activity. They do not activate reporter genes like estradiol or block the effect of estradiol like tamoxifen. Instead, compounds of this class produce a synergistic activation of reporter genes in the presence of estradiol (E2). Alone, the NRRP compounds have little to none of the agonist or antagonist effects of estradiol. However, the combination of E2 and an NRRP regulates two main classes of genes. One class of genes was synergistically activated by the NRRP/E2 combinations. The other class of genes was termed reprogrammed genes, because they are not regulated by ERα unless an NRRP is added in the presence of physiological concentrations of E2. The effect of the NRRPs is mediated through ER, because they did not result in synergism with estradiol when ER was not present, or they were inhibited by an ER antagonist or blocked by a GPER1 antagonist.

These compounds alone or in combination with estrogens can be used for multiple indications, as they result in modification of estrogen's activity. Unlike estradiol alone, the combination of NRRPs with estradiol resulted in no breast cancer or uterine cancer cell proliferation, suggesting their use will augment beneficial estrogen activity and reduce the risk of MHT-associated breast and uterine cancer.

Other uses and advantages of the various embodiments described herein will be apparent to those skilled in the art upon review of the following disclosure.

### BRIEF DESCRIPTION OF THE DRAWINGS

The features and advantages of the present invention will become apparent from a consideration of the following detailed description presented in connection with the accompanying drawings in which:

FIGS. 1-3 show the structure-activity relationship for several chalcone derivatives alone and in combination with E2 in ERE-TK-Luc-transfected U2OS cells.

FIG. 4 shows MCF-7 cell proliferation for several chalcone derivatives alone and in combination with E2.

FIG. 5 shows KRT19 and FGR expression levels in U2OS-ERα cells for several chalcone derivatives.

FIG. 6 shows luciferase activity in ERE-TK-Luc-transfected Ishikawa cells for 2′,3′,4′-trihydroxy chalcone and compound A9 with and without E2.

FIG. 7 shows luciferase activity in NKG2E-TK-Luc-transfected U2OS-ERβ Cells in the presence of doxycycline and several chalcone derivatives.

FIG. 8 provides the structures of several chalcone derivatives.

FIGS. 9 and 10 show luciferase activity in NKG2E-TK-Luc-transfected U2OS-ERβ Cells in the presence of doxycycline and several chalcone derivatives.

FIG. 11 shows the results of a cell proliferation assay in MC7 cells incubated with several chalcone derivatives with and without E2.

### DETAILED DESCRIPTION OF THE INVENTION

Described herein are nuclear receptor reprogramming compounds. In some embodiments, the nuclear receptor reprogramming compound is not an agonist, an antagonist or a mixed agonist/antagonist of estrogen receptor (ER) and/or the compound modulates estradiol (E2) activity only when E2 is present at a physiological level. In some embodiments, the compound has a structure of Formula I, or a pharmaceutically acceptable salt thereof, wherein the compound of Formula I is:

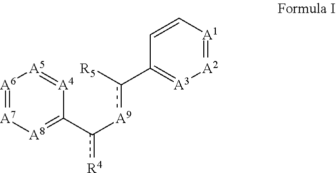

wherein,
each [symbol] is a single or double bond;
   A¹, A², and A³ are independently CR¹ or N,
   A⁴ is CR⁶ or N;
   A⁵, A⁶, and A⁷ are independently CR² or N,
   A⁸ is CR⁷ or N,
   A⁹ is CR³, CHR³, N, or NR³,
   each R¹ is independently H, halo, C₁-C₄ alkyl, OH, NH₂, or O—C₁-C₄ alkyl;
   each R² is independently H or OH,
   R³ is H or C₁-C₄ alkyl,
   R⁴ is O or OH, or together with R⁷ forms a pyrido or pyrimidino ring to form a quinoline, isoquinoline or quinazoline bicyclic ring system;
   R⁵ is H or, together with R⁶, forms an —O— bridge to form a benzopyran bicyclic ring system
   R⁶ is H or HO, or together with R⁵ forms an —O— bridge to form a benzopyran bicyclic ring; and
   R⁷ is H, OH, or together with R⁴ forms a pyrido or pyrimidine ring to form a quionline, isoquinoline, or qunazoline bicyclic ring system.

In some embodiments of the compound of the compound or salt of Formula I, each R¹ is independently H, F, CH₃, OH, NH₂, or O—CH₃.

In some embodiments of the compound or salt of Formula I, A⁴ is CH, A⁵ is CH, A⁶ is CH or COH, A⁷ is CH or COH, A⁸ is CH or COH.

In some embodiments of the compound or salt of Formula I, the [symbol] joined to R⁴ is a double bond and R⁴ is O.

In some embodiments of the compound or salt of Formula I, the [symbol] joined to A⁹ is a double bond.

In some embodiments of the compound or salt of Formula I, one and only one of A¹, A², and A³ is N, and the other two are CR¹.

In some embodiments of the compound or salt of Formula I, each R¹ is H.

In some embodiments of the compound or salt of Formula I, at least one R¹ is OH and the other R¹ is H or OH.

In some embodiments of the compound or salt of Formula I, one R¹ is OH and the other R¹ is H.

In some embodiments of the compound or salt of Formula I, A³ is N, A² is CH, and A³ is C—OH.

In some embodiments of the compound or salt of Formula I, at least one of A⁶, A⁷, or A⁸ is N.

In some embodiments of the compound or salt of Formula I, only one of A⁶, A⁷ and A⁸ is N.

In some embodiments of the compound or salt of Formula I, A¹ is C—OH.

In some embodiments of the compound or salt of Formula I, A² and A³ are CH.

In some embodiments of the compound or salt of Formula I, A9 is CR³ and R³ is H or C₁-C₄ alkyl.

In some embodiments of the compound or salt of Formula I, R³ is CH₃.

In some embodiments of the compound or salt of Formula I, the [symbol] joined to A⁹ is a single bond.

In some embodiments of the compound or salt of Formula I, A⁹ is CHR³.

In some embodiments of the compound or salt of Formula I, R³ is H or CH₃.

In some embodiments of the compound or salt of Formula I, A⁹ is NH.

In some embodiments of the compound or salt of Formula I, the [symbol] joine to R⁴ is a single bond.

In some embodiments of the compound or salt of Formula I, R⁴ is OH.

In some embodiments of the compound or salt of Formula I, R⁴ and R⁷ together form a pyrido or pyrimidino ring fused with the adjacent benzo ring.

In some embodiments of the compound or salt of Formula I, R⁴ and R⁷ together form a pyrido ring fused to the adjacent benzo ring to form a quinoline or an isoquinoline.

In some embodiments of the compound or salt of Formula I, R⁴ and R⁷ together form a pyrimidino ring fused with the adjacent benzo ring to form a quinazoline.

In some embodiments of the compound or salt of Formula I, A⁶ and A⁷ are C—OH.

In some embodiments of the compound or salt of Formula I, A⁴ and A⁵ are CH.

In some embodiments of the compound or salt of Formula I, R⁵ and R⁶ together form an —O— bridge.

In some embodiments of the compound or salt of Formula I, the compound is one of the following:

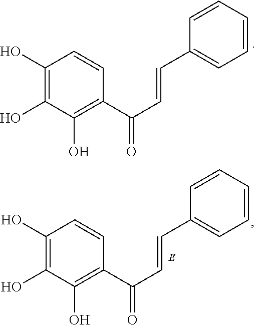 [SMILES: O=C(/C=C/c1ccccc1)c1ccc(O)c(O)c1O ; O=C(/C=C/c1ccccc1)c1ccc(O)c(O)c1O]

wherein “E” indicates the vinyl bond is entgegen.

In some embodiments of the compound or salt of Formula I, the compound is:

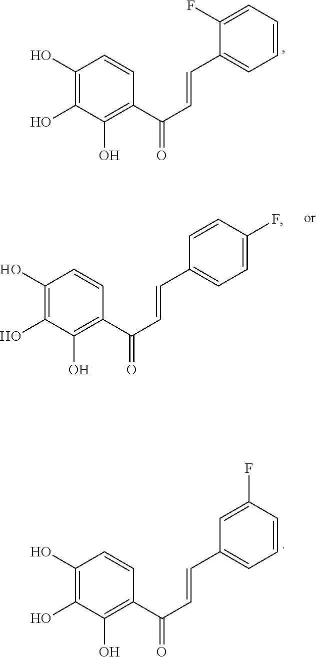 [SMILES: O=C(/C=C/c1ccccc1F)c1ccc(O)c(O)c1O ; O=C(/C=C/c1ccc(F)cc1)c1ccc(O)c(O)c1O ; "or" ; O=C(/C=C/c1cccc(F)c1)c1ccc(O)c(O)c1O]

In some embodiments of the compound of Formula I, the compound is:

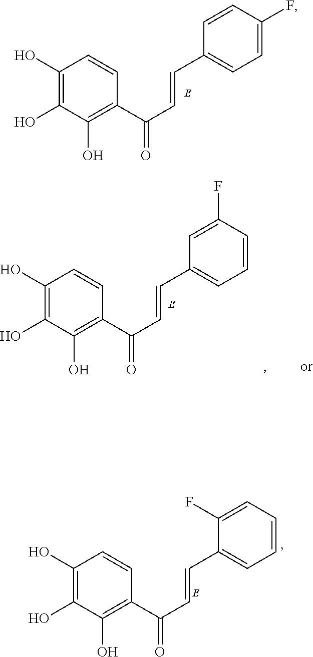 [SMILES: O=C(/C=C/c1ccc(F)cc1)c1ccc(O)c(O)c1O ; O=C(/C=C/c1cccc(F)c1)c1ccc(O)c(O)c1O ; "or" ; O=C(/C=C/c1ccccc1F)c1ccc(O)c(O)c1O]

wherein “E” indicates the vinyl bond is entgegen.

In some embodiments of the compound of Formula I, the compound is:

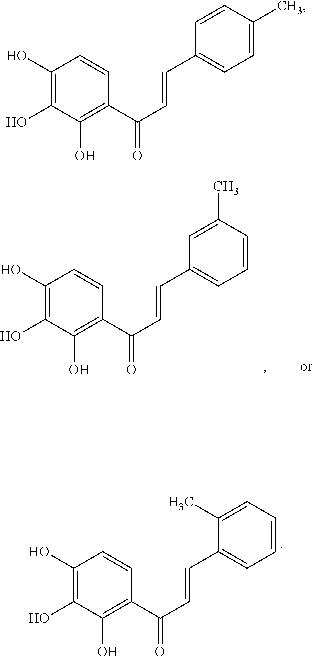 [SMILES: Cc1ccc(/C=C/C(=O)c2ccc(O)c(O)c2O)cc1 ; Cc1cccc(/C=C/C(=O)c2ccc(O)c(O)c2O)c1 ; "or" ; Cc1ccccc1/C=C/C(=O)c1ccc(O)c(O)c1O]

In some embodiments of the compound of Formula I, the compound is:

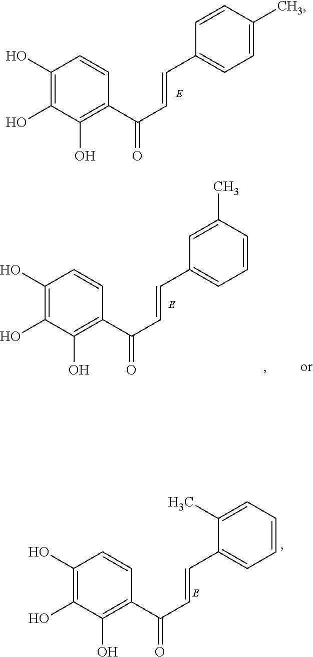 [SMILES: Cc1ccc(/C=C/C(=O)c2ccc(O)c(O)c2O)cc1 ; Cc1cccc(/C=C/C(=O)c2ccc(O)c(O)c2O)c1 ; "or" ; Cc1ccccc1/C=C/C(=O)c1ccc(O)c(O)c1O]

wherein “E” indicates the vinyl bond is entgegen.

In some embodiments of the compound of Formula I, the compound is:

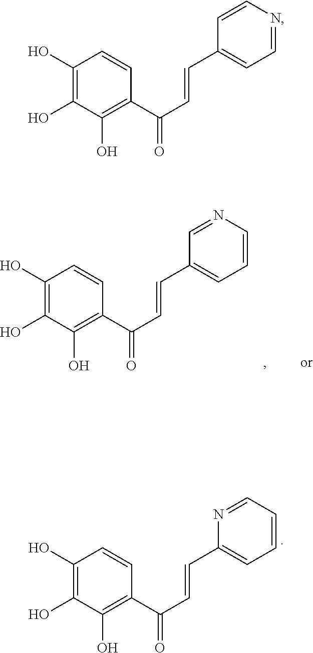 [SMILES: O=C(/C=C/c1ccncc1)c1ccc(O)c(O)c1O ; O=C(/C=C/c1cccnc1)c1ccc(O)c(O)c1O ; "or" ; O=C(/C=C/c1ccccn1)c1ccc(O)c(O)c1O]

In some embodiments of the compound of Formula I, the compound is:

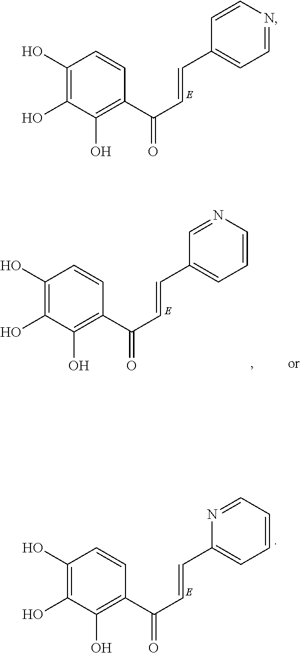 [SMILES: O=C(/C=C/c1ccncc1)c1ccc(O)c(O)c1O ; O=C(/C=C/c1cccnc1)c1ccc(O)c(O)c1O ; "or" ; O=C(/C=C/c1ccccn1)c1ccc(O)c(O)c1O]

In some embodiments of the compound of Formula I, the compound is:

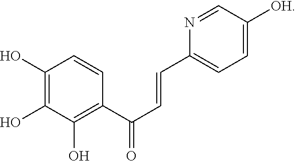 [SMILES: O=C(/C=C/c1ccc(O)cn1)c1ccc(O)c(O)c1O]

In some embodiments of the compound of Formula I, the compound is

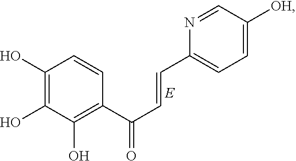 [SMILES: O=C(/C=C/c1ccc(O)cn1)c1ccc(O)c(O)c1O]

wherein “E” indicates the vinyl bond is entgegen.

In some embodiments of the compound of Formula I, the compound is:

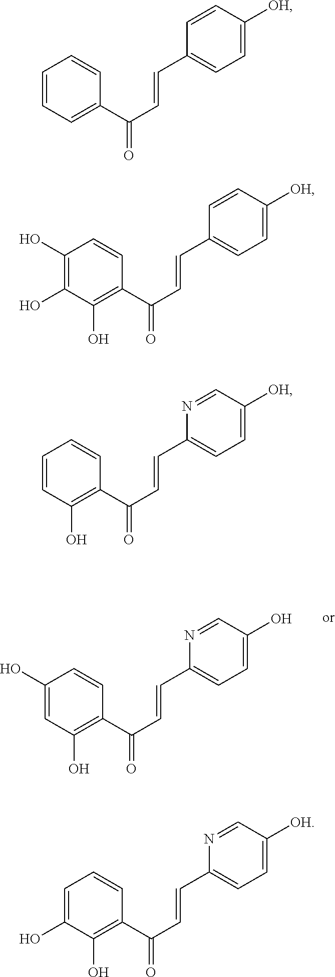 [SMILES: O=C(/C=C/c1ccc(O)cc1)c1ccccc1 ; O=C(/C=C/c1ccc(O)cc1)c1ccc(O)c(O)c1O ; O=C(/C=C/c1ccc(O)cn1)c1ccccc1O ; O=C(/C=C/c1ccc(O)cn1)c1ccc(O)cc1O ; "or" ; O=C(/C=C/c1ccc(O)cn1)c1cccc(O)c1O]

In some embodiments of the compound of Formula I, the compound is:

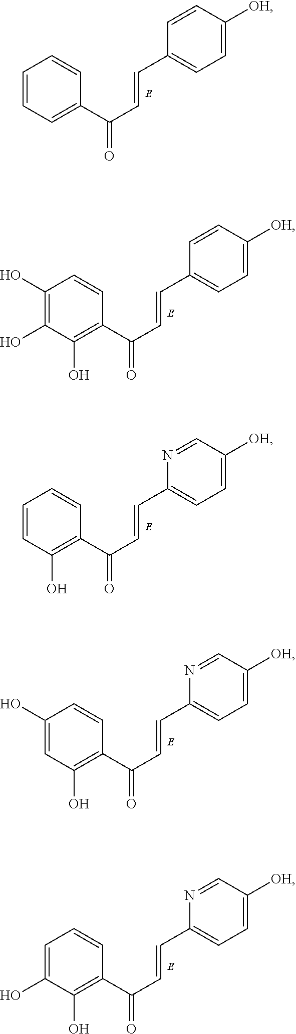 [SMILES: O=C(/C=C/c1ccc(O)cc1)c1ccccc1 ; O=C(/C=C/c1ccc(O)cc1)c1ccc(O)c(O)c1O ; O=C(/C=C/c1ccc(O)cn1)c1ccccc1O ; O=C(/C=C/c1ccc(O)cn1)c1ccc(O)cc1O ; O=C(/C=C/c1ccc(O)cn1)c1cccc(O)c1O]

wherein “E” indicates the vinyl bond is entgegen.

In some embodiments of the compound of Formula I, the compound is:

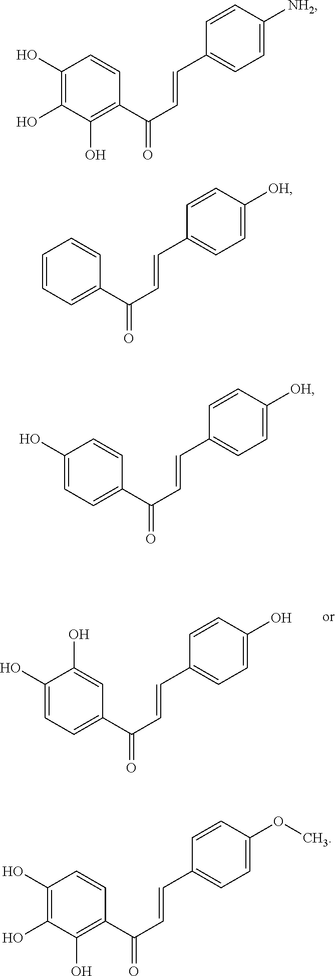 [SMILES: Nc1ccc(/C=C/C(=O)c2ccc(O)c(O)c2O)cc1 ; O=C(/C=C/c1ccc(O)cc1)c1ccccc1 ; O=C(/C=C/c1ccc(O)cc1)c1ccc(O)cc1 ; O=C(/C=C/c1ccc(O)cc1)c1ccc(O)c(O)c1 ; "or" ; COc1ccc(/C=C/C(=O)c2ccc(O)c(O)c2O)cc1]

In some embodiments of the compound of Formula I, the compound is:

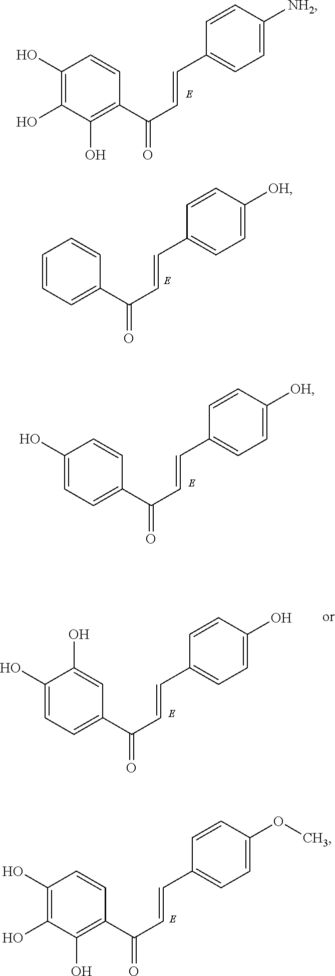 [SMILES: Nc1ccc(/C=C/C(=O)c2ccc(O)c(O)c2O)cc1 ; O=C(/C=C/c1ccc(O)cc1)c1ccccc1 ; O=C(/C=C/c1ccc(O)cc1)c1ccc(O)cc1 ; O=C(/C=C/c1ccc(O)cc1)c1ccc(O)c(O)c1 ; "or" ; COc1ccc(/C=C/C(=O)c2ccc(O)c(O)c2O)cc1]

wherein E indicates the vinyl bond is entgegen.

In some embodiments of the compound of Formula I, the compound is:

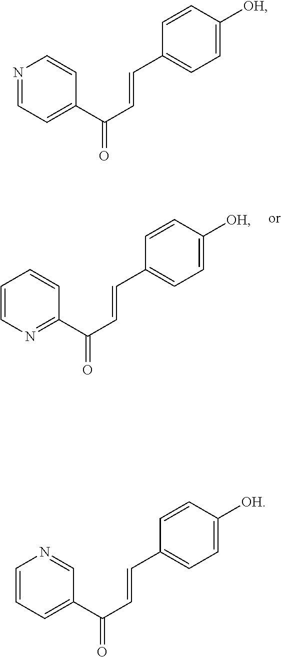 [SMILES: O=C(/C=C/c1ccc(O)cc1)c1ccncc1 ; O=C(/C=C/c1ccc(O)cc1)c1ccccn1 ; "or" ; O=C(/C=C/c1ccc(O)cc1)c1cccnc1]

In some embodiments of the compound of Formula I, the compound is:

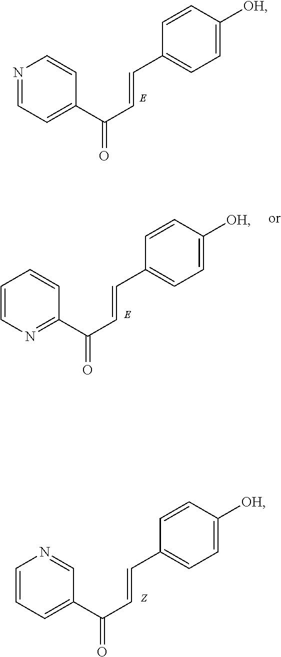 [SMILES: O=C(/C=C/c1ccc(O)cc1)c1ccncc1 ; O=C(/C=C/c1ccc(O)cc1)c1ccccn1 ; "or" ; O=C(/C=C/c1ccc(O)cc1)c1cccnc1]

wherein “E” indicates the vinyl bond is entgegen and “Z” indicates the vinyl bond is zusammen.

In some embodiments of the compound of Formula I, the compound is:

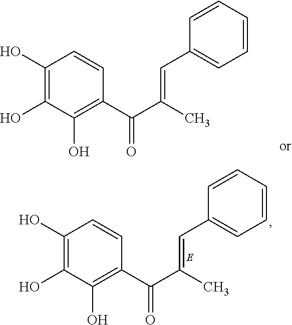 [SMILES: C/C(=C\c1ccccc1)C(=O)c1ccc(O)c(O)c1O ; C/C(=C\c1ccccc1)C(=O)c1ccc(O)c(O)c1O]

wherein “E” indicates the vinyl bond is entgegen.

In some embodiments of the compound of Formula I, the compound is:

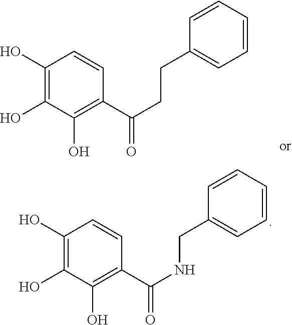 [SMILES: O=C(CCc1ccccc1)c1ccc(O)c(O)c1O ; O=C(NCc1ccccc1)c1ccc(O)c(O)c1O]

In some embodiments of the compound of Formula I, the compound is:

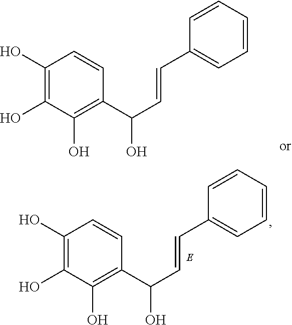 [SMILES: Oc1ccc(C(O)/C=C/c2ccccc2)c(O)c1O ; Oc1ccc(C(O)/C=C/c2ccccc2)c(O)c1O]

wherein E indicates the vinyl bond is entgegen.

In some embodiments of the compound of Formula I, the compound is:

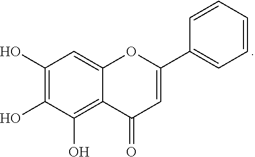 [SMILES: O=c1cc(-c2ccccc2)oc2cc(O)c(O)c(O)c12]

In some embodiments of the compound of Formula I, the compound is:

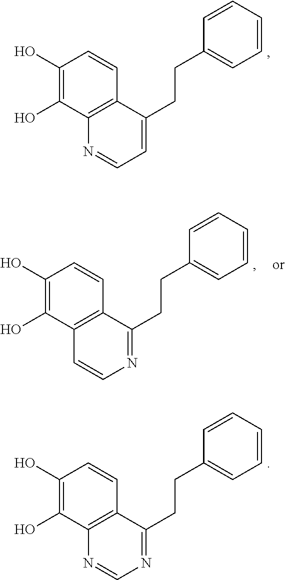 [SMILES: Oc1ccc2c(CCc3ccccc3)ccnc2c1O ; Oc1ccc2c(CCc3ccccc3)nccc2c1O ; Oc1ccc2c(CCc3ccccc3)ncnc2c1O]

Compounds of Formula I and pharmaceutically acceptable salts thereof are NRRP compounds, enhancing the in vitro activity of estrogen receptor alpha (ERα) in the presence of estradiol (E2) but neither agonizing nor antagonizing ERα in the absence of E2, and reprogramming one or more genes under the control of estrogen. Compounds of Formula I have utility in the treatment of one or more disorders as discussed in more detail herein below.

Some embodiments described herein comprise a pharmaceutical composition comprising a compound of Formula I, or a pharmaceutically acceptable salt thereof, and at least one pharmaceutically acceptable excipient. In some embodiments, the pharmaceutical composition may further comprise a member of the group consisting of estradiol (E2), one or more estrogen receptor agonists, one or more estrogen receptor antagonists, one or more mixed estrogen receptor agonist/antagonists, one or more selective estrogen receptor modulators (SERMs), one or more progestogens, one or more glucocorticoids, and one or more androgens.

### Synthesis

Compounds of the invention may be obtained as follows.

Variation of the “A” ring: Compounds. Compounds of the formula:

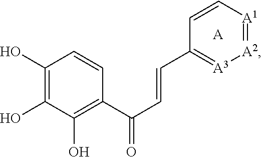

wherein:
“A” indicates the A ring of a chalcone;
A¹, A², and A³ are independently CR¹ or N; and
each R¹ is independently H, halo, C₁-C₄ alkyl, OH, NH₂, or O—C₁-C₄ alkyl, can be synthesized according to the following general method:

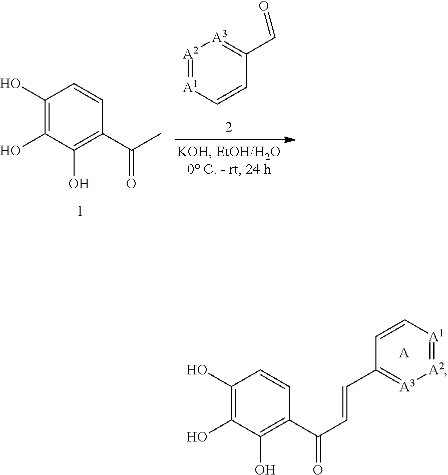

wherein A¹, A², and A³ have the meanings defined for compounds of Formula I. Compounds of formula 1 and 2 are commercially available. For example, following this procedure, the “A” ring may be explored as follows:

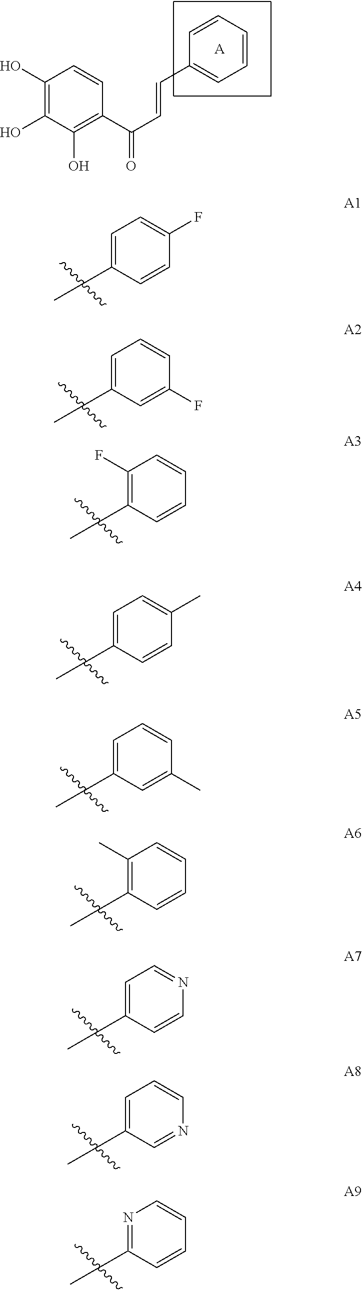

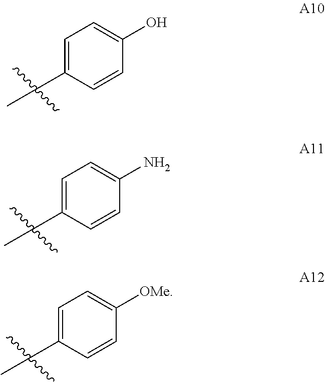 [SMILES: *c1ccc(O)cc1 ; *c1ccc(N)cc1 ; *c1ccc(OC)cc1]

Compounds of the formula:

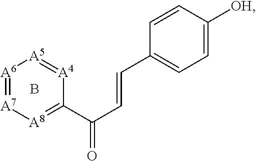

wherein A⁴-A⁸ have the definitions in Formula I and “B” indicates the B ring of a chalcone, by the following reaction:

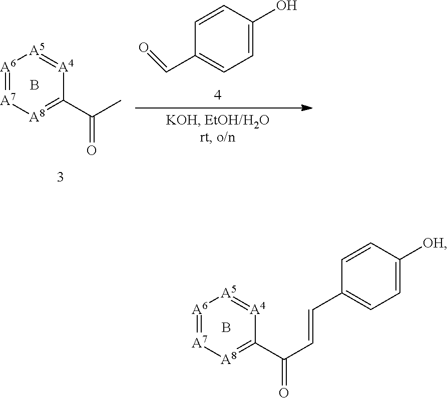

wherein A⁴-A⁸ have the meanings defined for compounds of Formula I. Compounds of formula 3 and 4 are commercially available. Following this procedure, the “B” ring may be explored as follows:

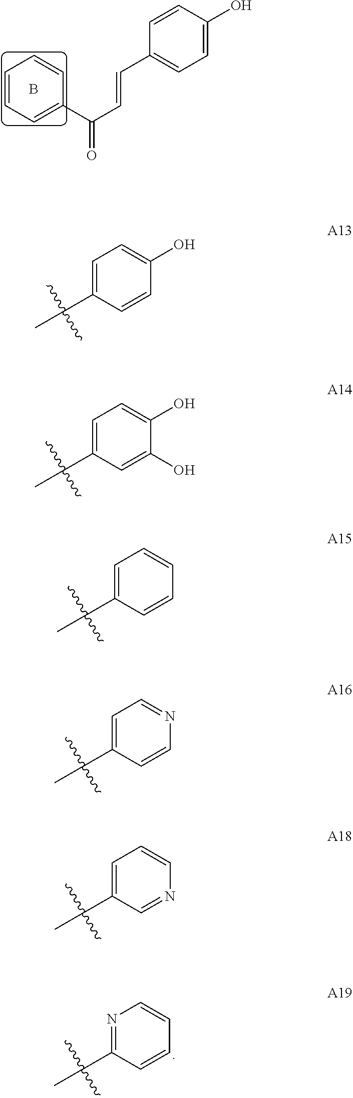

Compounds having a nitrogen-containing linker group between the A and B rings may be synthesized by the following synthetic reaction scheme:

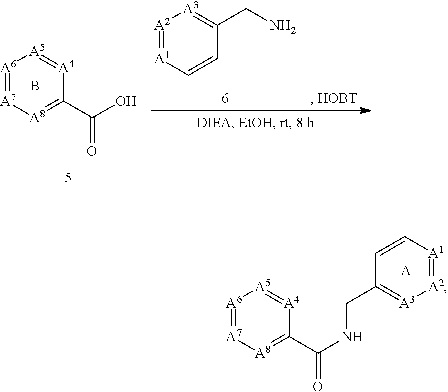

wherein A¹ -A⁸ are defined in formula I. Compounds 5 and 6 are commercially available.

In some embodiments, in which one of A⁴-A⁸ is OH, one or more of the hydroxyl groups may be methylated, in which case a second methyl-group removal step may be employed to produce the OH group. For example, the following reaction scheme results in compound A21:

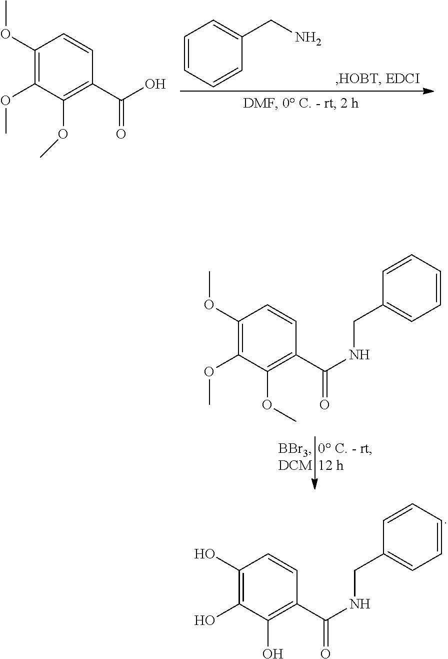

Conformationally restricted compounds like compounds A24 can be made by the following reaction:

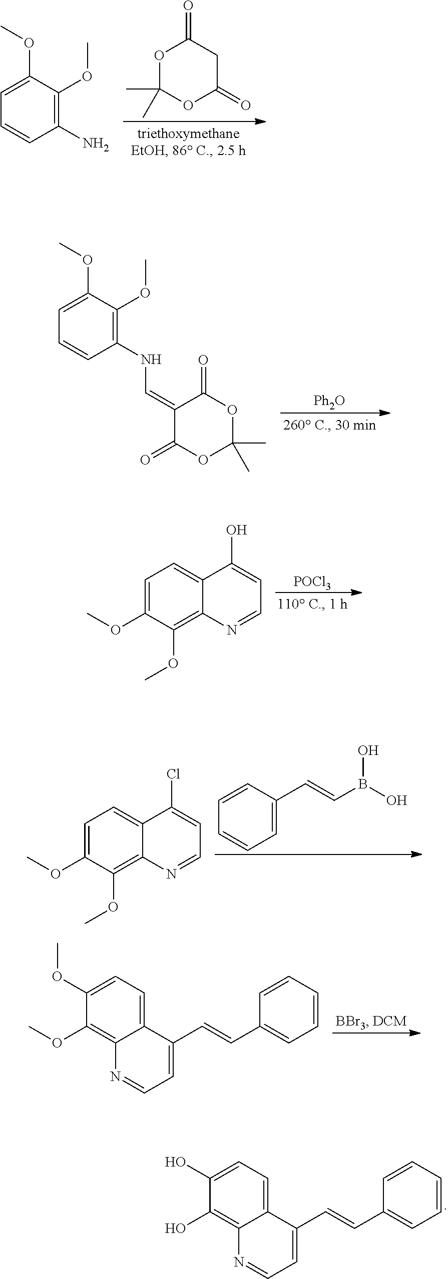

### Pharmaceutical Compositions

Pharmaceutical compositions described herein comprise. At least one of the pharmaceutically acceptable ingredients may include one or more ingredients that do not naturally occur with the novel compounds disclosed herein, a salt or a solvate thereof. Pharmaceutically acceptable ingredients that do not naturally occur with the novel compounds disclosed herein, a salt or a solvate thereof, in nature may include sterile, isotonic, or pyrogen free excipients.

A “pharmaceutically acceptable” ingredient is an ingredient that is compatible with the estrogenic compounds as described herein and with other ingredients of the composition and is suitable for administration to a patient. Additional ingredients may include carriers, diluents, absorption enhancers, stabilizers, preservatives, or other active or inactive ingredients. At least one of the additional ingredients may be an ingredient that does not occur naturally with the NRRP compound as described herein in nature. At least one of the additional ingredients may be an ingredient other than water. In some embodiments, the pharmaceutical composition may be sterile, pyrogen free, and/or isotonic. In some embodiments, the pharmaceutical composition is sterile or pyrogen free. In some embodiments, the pharmaceutical composition is sterile and pyrogen free. In some preferred embodiments, the pharmaceutical composition is sterile, pyrogen free and isotonic.

In some embodiments, the pharmaceutical composition may be an estrogenic composition. The estrogenic composition comprises an estrogenically effective amount of the NRRP compound as described herein and an additional ingredient. The additional ingredient may be an excipient. The excipient may comprise at least one compound that does not occur naturally with the NRRP compound in nature. In particular, the excipient may comprise at least one compound that does not naturally occur with the NRRP compound as described herein in humans. In some embodiments the excipient may comprise at least one compound other than water. In some embodiments, the additional compound may be a salt or other ingredient at a concentration sufficient for the composition to be isotonic. In some embodiments, the additional ingredient may be a flavor or sweetener not found with the NRRP compound as described herein in nature. In some embodiments, the estrogenic composition may be sterile, pyrogen free, and/or isotonic.

Pharmaceutically acceptable salts may be any salt of an estrogenic compound disclosed herein having suitable solubility in an aqueous solvent of appropriate pH. Remington's, 20th Ed., published 2000, pp. 704-719 provides methods for determining appropriate pharmaceutically acceptable salts. For example, suitable salts may be selected from Table 38-2, p. 704 of Remington's. The pharmaceutically acceptable salt may be prepared by dissolving the estrogenic compound in a suitable solvent and adding a suitable acid or base, or suitable counter-acid or counter-base, as the case may be, to the solution, and separating the salt form of the estrogenic compound from the solution.

Pharmaceutical compositions, in particular estrogenic compositions, may be formulated for a variety of routes of administration, such as oral, intranasal, intrapulmonary (e.g., for inhalation), intravenous, subcutaneous, transdermal, sublingual, buccal, intraperitoneal, or intrathecal administration. Pharmaceutical compositions may comprise one or more enhancers to assist in the transport of the NRRP compound as described herein against one or more external or internal physiological barriers, such as a pulmonary epithelial barrier or a blood brain barrier.

Suitable pharmaceutically acceptable excipients may include the following types of excipients: diluents, lubricants, binders, disintegrants, fillers, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents.

### Estrogenically Effective Doses

The effective dose of an NRRP compound may vary depending upon a variety of factors, including the route of administration, the age and condition of the patient in need of estrogenic treatment, co-administration with other drugs or compounds, etc. In general, an NRRP compound as described herein is effective in vitro at nanomolar or micromolar concentrations. Effective daily doses of an NRRP compound as described herein may be in the range of 0.01 mg to 1000 mg per day. The effective daily dose may be divided into two or more divided doses, e.g., 1, 2, 3, 4, 5, 6, or more divided doses. Where the estrogenic composition is administered as an infusion, the effective daily dose may be administered as a continuous infusion over a course of hours, e.g., 1-24 hours. An effective dose of may be similar to that of an NRRP compound as described herein, but may be scaled to account for the greater molecular weight of the compared to an NRRP compound as described herein at the compound's relative bioactivity, pharmacokinetics and pharmacodynamics, which one of skill in the art knows how to determine by art-recognized methods.

One skilled in art of pharmaceutical formulation and compounding possesses the knowledge and skill to select suitable pharmaceutically acceptable carriers and excipients in appropriate amounts for the use with an NRRP compound as described herein. In addition, there are a number of resources available those skilled in the art, which describe pharmaceutically acceptable carriers and excipients and may be useful in selecting suitable pharmaceutically acceptable carriers and excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The compositions of estrogenic compounds as described herein may be prepared using techniques and methods known to those skilled in the art. Some methods commonly used in the art are described in Remington's Pharmaceutical Sciences, 20^{th} Ed., (Mack Publishing Company (2000)).

In some embodiments, estrogenic compositions may comprise an NRRP compound as described herein and one or more pharmaceutically acceptable carriers or excipients. The composition may be prepared and packaged in bulk form wherein an effective amount of a compound of the disclosure can be extracted and then given to a subject, such as with powders or syrups. Alternatively, the composition may be prepared and packaged in unit dosage form wherein each physically discrete unit contains an effective amount of an NRRP compound as described herein.

An NRRP compound as described herein, and a pharmaceutically acceptable carrier or excipient(s), may be formulated into a dosage form adapted for administration to a subject by a desired route of administration. For example, dosage forms include those adapted for (1) oral administration, such as tablets, capsules, caplets, pills, troches, powders, syrups, elixirs, suspensions, solutions, emulsions, sachets, and cachets; and (2) parenteral administration, such as sterile solutions, suspensions, and powders for reconstitution. Suitable pharmaceutically acceptable carriers or excipients may vary depending upon the particular dosage form chosen. In addition, suitable pharmaceutically acceptable carriers or excipients may be chosen for a particular function that they may serve in the composition. For example, certain pharmaceutically acceptable carriers or excipients may be chosen for their ability to facilitate the production of uniform dosage forms. Certain pharmaceutically acceptable carriers or excipients may be chosen for their ability to facilitate the production of stable dosage forms. Certain pharmaceutically acceptable carriers or excipients may be chosen for their ability to facilitate the carrying or transporting of a compound disclosed herein, once administered to the subject, from one organ or portion of the body to another organ or another portion of the body. Certain pharmaceutically acceptable carriers or excipients may be chosen for their ability to enhance patient compliance.

In some embodiments, estrogenic an NRRP compound as described herein compositions may be formulated for parenteral administration. Compositions adapted for parenteral administration include aqueous and non-aqueous sterile injection solutions which may contain antioxidants, buffers, bacteriostats, and solutes that render the formulation isotonic with the blood of the intended recipient; and aqueous and non-aqueous sterile suspensions that may include suspending agents and thickening agents. The compositions may be presented in unit-dose or multi-dose containers, for example sealed ampoules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets. Parenteral formulations may be sterile, pyrogen-free, or both. Parenteral formulations may be isotonic.

### Oral

The estrogenic composition may be an oral estrogenic composition comprising an NRRP compound as described herein and at least one excipient suitable for oral administration. The at least one excipient suitable for oral administration may comprise a compound that does not occur naturally with an NRRP compound as described herein in nature. The at least one excipient suitable for oral administration may comprise at least one compound other than water. Various dosage forms may be prepared, such as tablets, capsules, caplets, troches, powders, emulsions, sachets, cachets, gel capsules, elixirs, pills, oral sprays, chewable tablets, sublingual tablets, films, or sprays, or buccal films or sprays.

In some embodiments, an NRRP compound as described herein may be formulated as a solid oral dosage form, such as a tablet or capsule comprising an effective amount of a compound of the disclosure and a diluent or filler. Suitable diluents and fillers include lactose, sucrose, dextrose, mannitol, sorbitol, starch (e.g., corn starch, potato starch, and pre-gelatinized starch), cellulose and its derivatives, (e.g., microcrystalline cellulose), calcium sulfate, and dibasic calcium phosphate. The oral solid dosage form may further comprise a binder. Suitable binders include starch (e.g., corn starch, potato starch, and pre-gelatinized starch) gelatin, acacia, sodium alginate, alginic acid, tragacanth, guar gum, povidone, and cellulose and its derivatives (e.g., microcrystalline cellulose). The oral solid dosage form may further comprise a disintegrant. Suitable disintegrants include crospovidone, sodium starch glycolate, croscarmellose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The compositions can also be prepared to prolong or sustain the release as, for example, by coating or embedding particulate material in polymers, wax, or the like.

An NRRP compound as described herein may also be combined with soluble polymers as targetable drug carriers. Such polymers can include polyvinylpyrrolidone, pyrancopolymer, polyhydroxypropylmethacrylamidephenol, polyhydroxyethylaspartam-idephenol, or polyethylene-oxidepolylysine substituted with palmitoyl residues. Furthermore, an NRRP compound as described herein may be combined with a class of biodegradable polymers useful in achieving controlled release of a drug, for example polylactic acid, polepsilon caprolactone, polyhydroxy butyric acid, polyorthoesters, polyacetals, polydihydropyrans, polycyanacrylates and cross-linked or amphipathic block copolymers of hydrogels.

In some embodiments, an NRRP compound as described herein may be formulated in a liquid oral dosage form. Oral liquids such as solution, syrups and elixirs can be prepared in dosage unit form so that a given quantity contains a predetermined amount of a compound disclosed herein. Syrups can be prepared by dissolving the compound of the disclosure in a suitably flavored aqueous solution, while elixirs are prepared through the use of a non-toxic alcoholic vehicle. Suspensions can be formulated by dispersing a compound disclosed herein in a non-toxic vehicle. Solubilizers and emulsifiers such as ethoxylated isostearyl alcohols and polyoxy ethylene sorbitol ethers, preservatives, flavor additives such as peppermint oil or other natural sweeteners or saccharin or other artificial sweeteners and the like can also be added.

### Intranasal

The estrogenic composition may be an intranasal estrogenic composition comprising an NRRP compound as described herein and at least one excipient suitable for intranasal administration. The at least one excipient suitable for intranasal administration may comprise at least one compound other than water. For example, the intranasal estrogenic composition may comprise one or more penetration enhancers, which increase absorption of an NRRP compound as described herein across the mucosa and/or increase bioavailability. In some embodiments, penetration enhancers may include mucolytic agents, degradative enzyme inhibitors and compounds which increase permeability of the mucosal cell membranes. Whether a given compound is an “enhancer” can be determined by comparing two formulations comprising a non-associated, small polar molecule as the drug, with or without the enhancer, in an in vivo or good model test and determining whether the uptake of the drug is enhanced to a clinically significant degree. The enhancer should not produce any problems in terms of chronic toxicity because in vivo the enhancer should be non-irritant and/or rapidly metabolized to a normal cell constituent that does not have any significant irritant effect. In some embodiments, the penetration enhancer may be an alkyl glycoside, e.g., an alkyl glycoside disclosed in U.S. Pat. No. 5,661,130, which is incorporated herein by reference in its entirety. One skilled in the art recognizes the need to achieve a suitable hydrophile-lipophile balance (HLB) number, which may be determined as disclosed in U.S. Pre-Grant Publication No. US2009/0047347, which is incorporated herein by reference in its entirety.

Intranasal estrogenic compositions of an NRRP compound as described herein may also include flavors or scents to cover the taste of an NRRP compound as described herein. Intranasal compositions may also include isotonizing agents to make the composition isotonic. Intranasal estrogenic compositions of an NRRP compound as described herein may also include stabilizing agents.

### Intrapulmonary

The estrogenic composition may be an intrapulmonary estrogenic composition comprising an NRRP compound as described herein and at least one excipient suitable for intranasal administration. The at least one excipient suitable for intrapulmonary administration may comprise at least one compound other than water. For example, the intrapulmonary composition may comprise one or more penetration enhancers, which increase the ability of an NRRP compound as described herein to cross the pulmonary epithelia into the blood stream.

Intrapulmonary estrogenic compositions may be administered to the lungs by inhalation, e.g., using an insufflator, aerosol inhaler, or a conventional or high efficiency nebulizer.

High efficiency nebulizers are inhalation devices that comprise a microperforated membrane through which a liquid solution is converted through electrical or mechanical means into aerosol droplets suitable for inhalation. High efficiency nebulizers can deliver a large fraction of a loaded dose to a patient. In some embodiments, the high efficiency nebulizer may also utilize one or more actively or passively vibrating microperforated membranes. In some embodiments, the high efficiency nebulizer may comprise one or more oscillating membranes. In some embodiments, the high efficiency nebulizer may comprise a vibrating mesh or plate with multiple apertures and optionally a vibration generator with an aerosol mixing chamber. In some such embodiments, the mixing chamber may function to collect (or stage) the aerosol from the aerosol generator.

In some embodiments, the high efficiency nebulizer may achieve lung deposition (deposited lung dose) of at least about 10% based on the nominal dose of an NRRP compound as described herein.

In some embodiments, the high efficiency nebulizer provides an NRRP compound as described herein lung deposition (deposited lung dose) of at least about 5% based on the nominal dose of an NRRP compound as described herein.

In accordance with the invention, in some embodiments, a nebulizer, such as a high efficiency nebulizer may be adapted or adaptable to operate in conjunction with a unit dosage form, such as an ampule or vial, which contains a single dose of an NRRP compound as described herein for estrogenic therapy. The unit dosage form comprises a container that contains an inhalation solution comprising an NRRP compound as described herein. The container is adapted to cooperate with the high efficiency nebulizer device in such a way as to permit administration of the nominal dose of the inhalation solution to a patient in need thereof. In some embodiments, the high efficiency nebulizer and the unit dosage form are configured so that they are useable together, but not with other devices or dosage forms. In some particular embodiments, the unit dosage form is configured such that it fits into a keyhole-like structure in the high efficiency nebulizer but will not operate with other nebulizer devices. In such embodiments, the high efficiency nebulizer is configured such that it will accept and properly operate with the unit dosage form containing an NRRP compound as described herein, but not with other dosage forms.

Suitable high efficiency nebulizers with perforated membranes are disclosed in U.S. Pat. Nos. 6,962,151, 5,152,456, 5,261,601, and 5,518,179, each of which is hereby incorporated by reference in its entirety. Suitable high efficiency nebulizers contain oscillatable membranes. Features of these high efficiency nebulizers are disclosed in U.S. Pat. Nos. 7,252,085; 7,059,320; 6,983,747, each of which is hereby incorporated by reference in its entirety.

Commercial high efficiency nebulizers are available from: PARI (Germany) under the trade name eFlow®; Aerogen, Ltd. (Ireland) under the trade names AeroNeb® Go and AeroNeb® Pro, AeroNeb® Solo, and other nebulizers utilizing the OnQ® nebulizer technology; Respironics (Murrysville, Calif.) under the trade names I-Neb®; Omron (Bannockburn, Ill.) under the trade name Micro-Air®; Activaero (Germany) under the trade name Akita®, and AerovectRx (Atlanta, Ga.) under the trade name AerovectRx®.

Conventional nebulizers include, for example jet nebulizers or ultrasonic nebulizers. Jet nebulizers generally utilize compressors to generate compressed air, which breaks the liquid medication into small breathable droplets, which form an aerosolized (atomized) mist. In some of these embodiments, when the patient breathes in, a valve at the top opens, which then allows air into the apparatus, thereby speeding up the mist generation; when the patient breathes out, the top valve closes, thereby slowing down the mist generation while simultaneously permitting the patient to breathe out through the opening of a mouthpiece flap.

Some conventional nebulizers are disclosed in U.S. Pat. Nos. 6,513,727, 6,513,519, 6,176,237, 6,085,741, 6,000,394, 5.957,389, 5,740,966, 5,549,102, 5,461,695, 5,458,136, 5,312,046, 5,309,900, 5,280,784, and 4,496,086, each of which is hereby incorporated by reference in its entirety.

Commercial conventional nebulizers are available from: PARI (Germany) under the trade names PARI LC Plus®, LC Star® and PARI-Jet® A & H Products, Inc. (Tulsa, Okla.) under the trade name AquaTower®; Hudson RCI (Temecula, Calif.) under the trade name AVA-NEB®; Intersurgical, Inc. (Liverpool, N. Y.) under the trade name Cirrus®; Salter Labs (Arvin, Calif.) under the trade name Salter 8900®; Respironics (Murrysville, Pa.) under the trade name Sidestream®; Bunnell (Salt Lake City, Utah) under the trade name Whisper Jet®; Smiths-Medical (Hyth Kent, UK) under the trade name Downdraft®, and DeVilbiss (Somerset, Pa.) under the trade name DeVilbiss®.

### Intravenous

The estrogenic composition may be an intravenous estrogenic composition comprising an NRRP compound as described herein and at least one excipient suitable for intravenous administration. The at least one excipient suitable for intravenous administration may comprise at least one compound other than water. Intravenous compositions of an NRRP compound as described herein are parenteral compositions intended for intravenous administration by injection or infusion. They may contain one or more isotonizing agents to make the compositions isotonic. They may be, and generally are, sterile, pyrogen free, or both.

### Subcutaneous

The estrogenic composition may be a subcutaneous estrogenic composition comprising an NRRP compound as described herein and at least one excipient suitable for subcutaneous administration. The at least one excipient suitable for subcutaneous administration may comprise at least one compound other than water. Subcutaneous compositions of an NRRP compound as described herein are parenteral compositions intended for injection under the skin. They may contain one or more isotonizing agents to make the compositions isotonic. They may be, and generally are, sterile, pyrogen free, or both.

### Transdermal

The estrogenic composition may be a transdermal estrogenic composition comprising an NRRP compound as described herein and at least one excipient suitable for transdermal administration. The at least one excipient suitable for transdermal administration may comprise at least one compound other than water. For example, the transdermal estrogenic composition may comprise one or more penetration enhancers, which increase the ability of an NRRP compound as described herein to cross the dermis into the blood stream. In addition, the transdermal composition may be delivered by a biasing mechanism, such as an iontophoresis device.

### Sublingual or Buccal

The estrogenic composition may be a sublingual or buccal estrogenic composition comprising an NRRP compound as described herein and at least one excipient suitable for sublingual or buccal administration. The at least one excipient suitable for sublingual or buccal administration may comprise at least one compound other than water.

### Intraperitoneal

The estrogenic composition may be an intraperitoneal estrogenic composition comprising an NRRP compound as described herein and at least one excipient suitable for intraperitoneal administration. The at least one excipient suitable for intraperitoneal administration may comprise at least one compound other than water. Intraperitoneal estrogenic compositions of an NRRP compound as described herein are parenteral compositions intended for administration to the peritoneum by injection or infusion. They may contain one or more isotonizing agents to make the compositions isotonic. They may be, and generally are, sterile, pyrogen free, or both.

### Intrathecal or Intracranioventricular

The estrogenic composition may be an intrathecal or intracranioventricular estrogenic composition comprising an NRRP compound as described herein and at least one excipient suitable for intrathecal or intracranioventricular administration. The at least one excipient suitable for intrathecal or intracranioventricular administration may comprise at least one compound other than a compound that occurs naturally with an NRRP compound as described herein in nature, e.g., water. Intrathecal or intracranioventricular estrogenic compositions of an NRRP compound as described herein are parenteral compositions intended for administration into the cerebrospinal fluid administration by injection or infusion. They may contain one or more isotonizing agents to make the compositions isotonic. They may be, and generally are, sterile, pyrogen free, or both.

### Other Routes of Administration

Although there have been shown and described preferred embodiments of the compositions and methods described herein, it will be readily apparent to those skilled in the art that modifications may be made thereto which do not exceed the scope of the appended claims. Therefore, the scope of the described compositions and methods is only to be limited by the following claims.

### Estrogenic Methods

Some embodiments described herein provide a compound of Formula I, or a pharmaceutically acceptable salt thereof, or a pharmaceutical composition described herein, for use in treatment of a condition or disease state in a patient in need of treatment with a nuclear receptor reprogramming compound. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

Some embodiments described herein provide for use of a compound of Formula I, or a pharmaceutically acceptable salt thereof, for manufacture of a medicament for use in treatment of a condition or disease in a patient in need of treatment with a nuclear receptor reprogramming compound. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

Some embodiments described herein provide a method of treating a patient in need of such treatment, comprising administering to the patient an effective amount of a compound of Formula I, or a pharmaceutically acceptable salt thereof, or a pharmaceutical composition described herein. In some embodiments, the condition or disease state is a development condition, a menstruation condition, a fertility condition, a gynecological disease, an autoimmune disorder, a menopause disorder, an aging disorder, or cancer. In some embodiments, the condition or disease state is Turner Syndrome, Kallman Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crone's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.

In light of the disclosure herein, one skilled in the art understands how to determine an estrogenically effective amount of an NRRP compound as described herein. Generally, an estrogenically effective amount, a therapeutically effective amount, or a prophylactically effective amount, of an NRRP compound as described herein may be determined, e.g., by inference from in vitro testing. One skilled in the art understands that an effective dose may be inferred from the in vitro half maximal modulating (inhibitory or activating) concentration of an NRRP compound as described herein. One skilled in the art understands that the effective dose in human patients will depend on the route of administration, the pharmacokinetics, etc. Taking these factors into consideration, an estrogenically effective dose of an NRRP compound as described herein may be in the range of 0.1 mg/kg to 150 mg/kg, e.g. 0.1 mg/kg to 1 mg/kg, 0.5 mg/kg to 5 mg/kg, 1 mg/kg to 10 mg/kg, 5 mg/kg to 50 mg/kg, 10 mg/kg to 100 mg/kg, or 50 mg/kg to 150 mg/kg; an effective daily dose of an NRRP compound as described herein may be some multiple of any of the values within these ranges, e.g. one to six (1 to 6) times the values within these ranges.

### Transitional Phrases

In some embodiments, descriptions of the compositions and methods described herein using the transitional word “comprising” indicates that the compositions or methods are “open” to additional ingredients, components or steps. It is intended that “comprising” subsume the more limiting transitional phrases “consisting essentially of” and “consisting of.” Thus, disclosure herein of matter following the transitional phrase “comprising” also fully discloses the same following the transitional phrases “consisting essentially of” or “consisting of.” The transitional phrase “consisting essentially of,” is of intermediate effect, indicating that the subject matter that follows consists only of the recited elements and such additional matter as does not materially affect the novel and basic properties of the claim or claim element. The transitional phrase “consisting of,” indicates that the subject matter that follows is limited to the recited steps or ingredients and is closed to other steps or ingredients not recited. Where a transitional phrase appears within a clause or a sub-clause following another transitional phrase, it is intended that the embedded transitional phrase affect only the phrase in which it appears. Where “a” or “an” is recited in the specification or claims, the plural is also intended unless the singular is explicitly recite (e.g., “a single,” “only one,” etc.).

### EXAMPLES

Pharmaceutical compositions and estrogenic methods disclosed herein may be further understood with reference to the following examples.

### Example 1

Analogs of 2′,3′,4′-trihydroxychalcone (CC7).

Analogs of parent CC7 were prepared, as set forth in the following Tables 1A-1C.

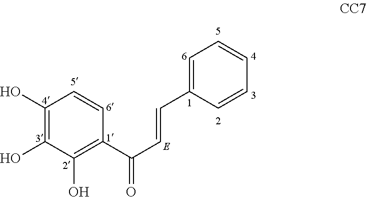 [SMILES: O=C(/C=C/c1ccccc1)c1ccc(O)c(O)c1O]

TABLE 1A
Examples A1-A11 are derivatives of CC7, i.e., 2′,3′,4′-trihydroxy-
chalcone or “THC,” with indicated changes to the “A” ring:

| | |
|---|---|
| | CC7 |
| 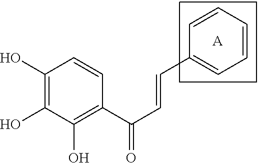 | |

| Example | A Ring |
|---|---|
| A1 | 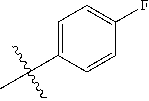 [SMILES: *c1ccc(F)cc1] |
| A2 | 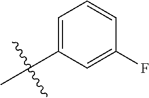 [SMILES: *c1cccc(F)c1] |
| A3 | 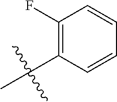 [SMILES: *c1ccccc1F] |
| A4 | 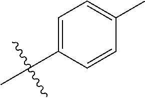 [SMILES: *c1ccc(C)cc1] |
| A5 | 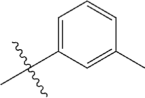 [SMILES: *c1cccc(C)c1] |
| A6 | 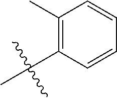 [SMILES: *c1ccccc1C] |
| A7 | 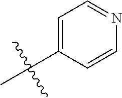 [SMILES: *c1ccncc1] |
| A8 | 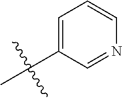 [SMILES: *c1cccnc1] |
| A9 | 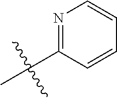 [SMILES: *c1ccccn1] |
| A10 | 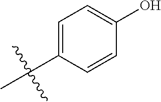 [SMILES: *c1ccc(O)cc1] |
| A11 | 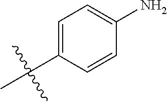 [SMILES: *c1ccc(N)cc1] |

TABLE 1B

| A13-A18 are derivatives of the following structure, with the indicated | |
|---|---|
| changes to the “B” ring: | |
| 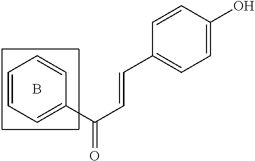 | |
| Example | B Ring |
| A13 | 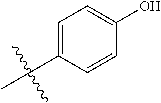 [SMILES: *c1ccc(O)cc1] |
| A14 | 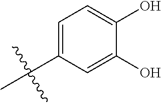 [SMILES: *c1ccc(O)c(O)c1] |
| A15 | 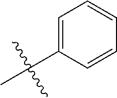 [SMILES: *c1ccccc1] |
| A16 | 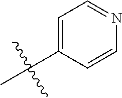 [SMILES: *c1ccncc1] |
| A17 | 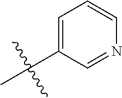 [SMILES: *c1cccnc1] |
| A18 | 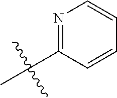 [SMILES: *c1ccccn1] |

TABLE 1C
Examples A20, A23, and A24 are variations on parent CC7.

| Example | Structure |
|---|---|
| A20 | 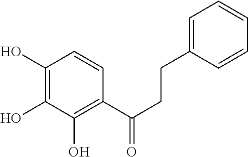 [SMILES: O=C(CCc1ccccc1)c1ccc(O)c(O)c1O] |
| A23 | 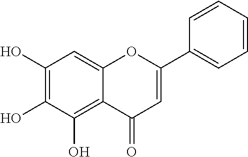 [SMILES: O=c1cc(-c2ccccc2)oc2cc(O)c(O)c(O)c12] |
| A24 | 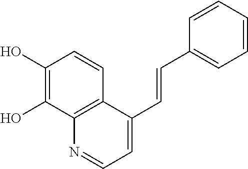 [SMILES: Oc1ccc2c(/C=C/c3ccccc3)ccnc2c1O] |

### Example 2

### Estrogen Receptor (ERα) Stimulation in U2OS Cells

Herein are described nuclear receptor reprogramming (NRRP) compounds, including those set forth in Tables 1A, 1B, and IC (“Test Compounds”), which are neither E2 agonists nor antagonists, nor do they result in mixed E2 agonist/antagonist activity. They do not activate reporter genes like E2. Nor do they block the effect of E2 on ERα like tamoxifen. Instead, these compounds produce synergistic activation of reporter genes in the presence of E2. Alone, the NRRP compounds have little or no estrogen-like agonist or antagonist effect on the ERE. However, despite this lack of activity in the absence of E2, in the presence of E2 the NRRPs act synergistically to regulate two main classes of genes. One class of genes is synergistically activated by the NRRP/E2 combinations. The other class of genes is termed reprogrammed genes because they are not regulated by ERα unless an NRRP is added to physiological concentrations of E2. The effect of the NRRP is mediated through the ER, because they did not result in synergism with E2 when the ER was not present.

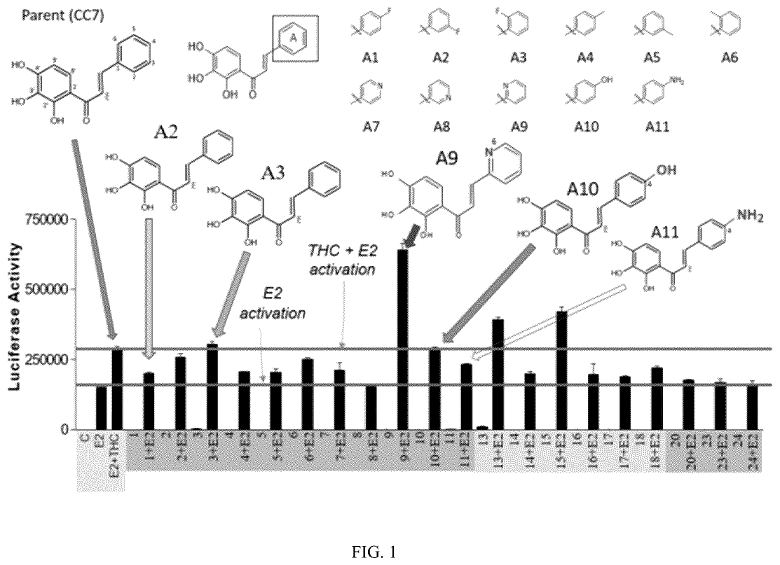

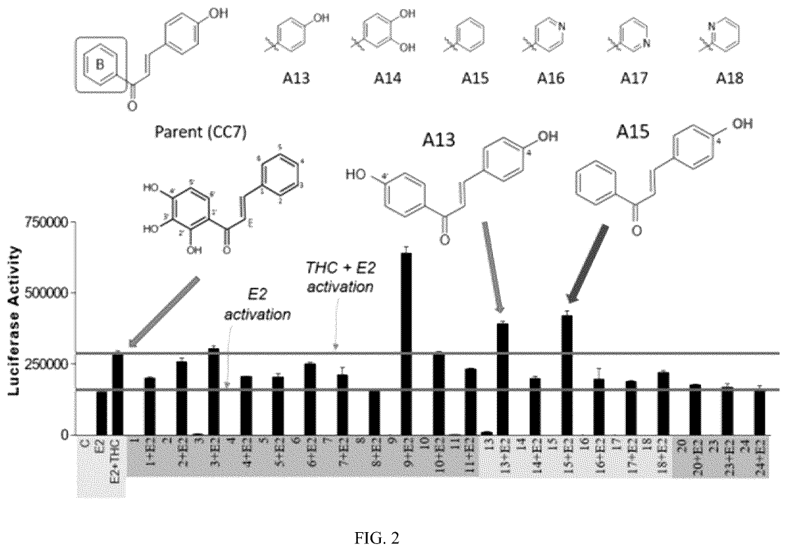

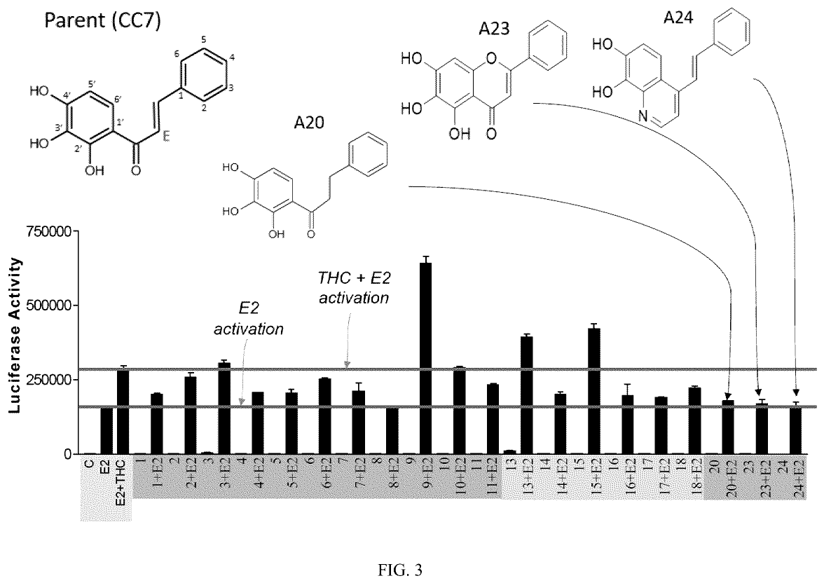

U2OS cells (wild type) were maintained in 5% charcoal-dextran stripped FBS. The cells were transfected with 3 μg of a plasmid containing the ERE upstream of the minimal thymidine kinase luciferase promoter (ERE-TK-Luc) and 1 μg of an ERα expression vector by electroporation as previously described (An et al. 2001). The resulting ERE-TK-Luc/ERα U2OS cells were incubated for 24 hours in the presence of medium (negative control), E2, and the Test Compounds, with and without E2. As expected, E2 alone activated ERE-TK-Luc. (See U.S. Pat. No. 7,482,029, which is incorporated by reference in its entirety.) See red horizontal line, FIGS. 1-3. As can be seen in FIGS. 1-3, No significant activation of ERE-TK-Luc occurred with the Test Compounds alone. In contrast, and unexpectedly, when combined with E2, the Test Compounds resulted in varying levels of synergistic activation of the ERE. These activities are considered synergistic because the activity of each of the Test Compounds plus E2 was greater than the sum of the activity of E2 plus the activity of the respective Test Compound.

### Example 3

### MCF-7 Cell Proliferation

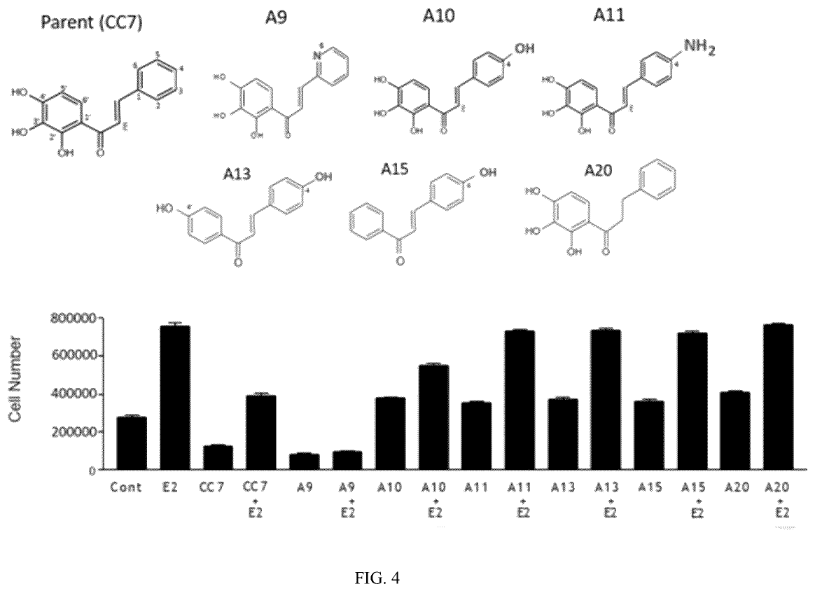

In order for long term menopausal hormone therapy to be a viable alternative to currently available therapeutic options it must not promote breast cancer. The growth-promoting properties of several compounds of the invention were studied in MCF-7 breast cancer cells, alone and in combination with E2. MCF-7 cells were incubated for 24 hr in the presence of medium (control), 1 nM F2, or 5 μM of each of the compounds depicted in FIG. 4 (CC7 and six analogs, A9, A10, A11, A13, A15, and A20). Viable cell counts were determined by flow cytometry.

MCF-7 cells were plated at a density of 50,000 cells per well in 6-well tissue culture plates in DMEM/F12 supplemented with 5% stripped FBS. The next day the cells were treated with vehicle or E2 in the absence and presence of 2′,3′,4′-THC or one of the six analogs in FIG. 4 for 7 days. The cells were then detached with trypsin, neutralized with media containing 5% FBS, and resuspended. Appropriate amounts of cell suspension were placed in ISOTON II diluent (Thermo Fisher Scientific, Waltham, MA, USA) and the cell numbers were then measured using a Coulter Counter (Beckman, Brea, CA, USA).

Flow cytometry was performed based on a previously described method (Pan et al. 2016). Briefly, the cells were plated at a density of 500,000 cells per well in 6-well tissue culture dishes in DMEM/F-12 supplemented with 5% stripped FBS for 48 h. The cultured medium was then replaced by serum-free DMEM/F12 for 24 h. The cells were then treated with vehicle, E2 without or with the 2′,3′,4′-THC or one of the six analogs in FIG. 4 for 24 h. The culture medium was then aspirated, and the cells were washed with PBS, detached with trypsin and collected by centrifugation at 1700 rpm for 5 min. The cell pellets were washed with ice cold PBS followed by centrifugation at 1700 rpm for 10 min at room temperature. The cell pellets were resuspended in 500 μL PBS containing 50 μg/mL propidium iodide, 0.1% of triton X-100, 0.1% of sodium citrate, and 10 μg/mL of RNase. The cell suspensions were then analyzed with a BD LSR II Flow Cytometer (BD Biosciences, San Jose, CA, USA) in the Flow Cytometry facility at University of California, Berkeley and the percentage of cells in cell cycle phases were determined by using FlowJo 7.6.5 (FlowJo, LLC, Ashland, OR, USA).

As can be seen in FIG. 4, E2 alone stimulated breast cancer cell proliferation. In contrast, the viable cell counts for CC7 and each of the six analogs were well below the cell counts for E2 alone. The cell counts for A9+E2 was well below the control, which was below the cell count for E2, while the cell counts for CC7+E2 and A10+E2 were well below the cell counts for E2 alone. These results show that CC7 and the six analogs do not stimulate MCF-7 breast cancer cell proliferation like E2. Additionally, addition of each of the six analogs to E2 does not enhance E2's stimulation of MCF-7 cell proliferation, and in some cases even inhibits E2's pro-stimulatory activity.

### Example 4

### Gene Expression Assays

Human U2OS (osteosarcoma) cells expressing a tetracycline-regulated ERα (U2OS-ERα) were prepared, characterized, and maintained as previously described (Tee et al., 2004). The cells were maintained in phenol red-free Gibco DMEM/F-12 (Thermo Fisher Scientific, Waltham, MA, USA) supplemented with 5% charcoal-dextran stripped fetal bovine serum (FBS, Gemini Bio Products, West Sacramento, CA, USA), 100 units/mL penicillin and streptomycin, 50 μg/mL Fungizone, and 2 mM of glutamine. To maintain stable transfected cells, 50 μg/mL hygromycin B and 500 μg/mL of zeocin (Invitrogen, Waltham, MA, USA) were included in culture media.

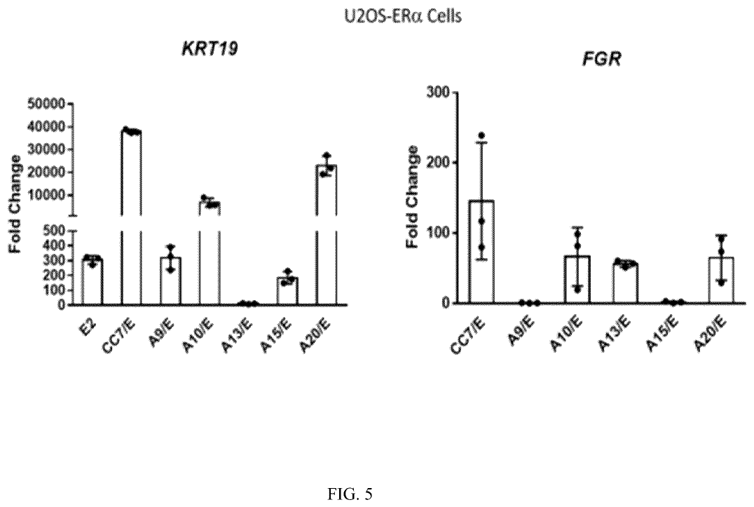

U2OS-ERα cells were treated with E2 in the absence or in combination with 2′,3′,4′-THC (CC7) and real-time PCR analysis was performed. E2 alone activated KRT19 (FIG. 5) and a large synergistic effect was observed with CC7. In contrast, “reprogrammed” gene FGR was activated by the combination of E2 and CC7 only, and not by either compound by itself.

Similar experiments were conducted with analogs of CC7, i.e., A9, A10, A13, A15, and A20. See FIG. 5 for results.

### Example 5

### Luciferase Activity in ERE-TK-Luc in Ishikawa Cells

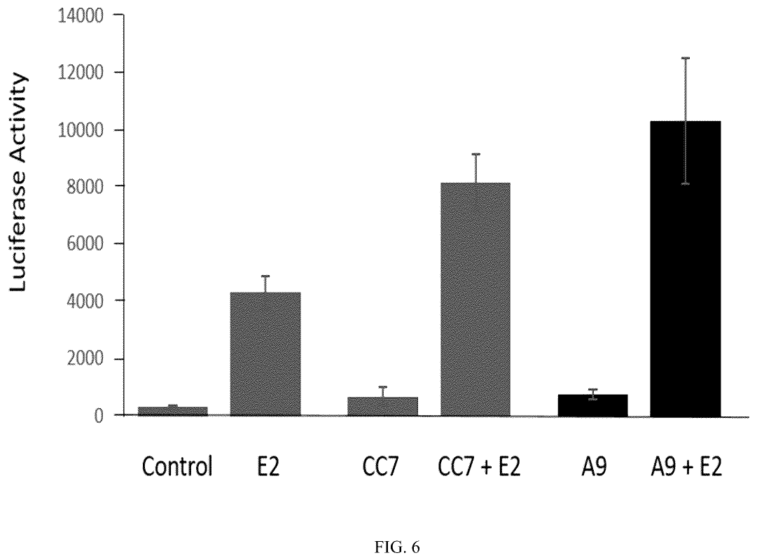

Ishikawa cells transfected with the ERE-TK-Luc reporter gene and ERα were incubated with E2 alone, CC7 alone, A9 alone, CC7+E2, or A9+E2. See FIG. 6. CC7 and A9 had no effect on the luciferase activity of the transfected cells, demonstrating that these compounds have no estrogenic effect by themselves. In combination with E2, however, CC7 and A9 each demonstrated synergism with E2, as can be seen in FIG. 6.

### Example 6

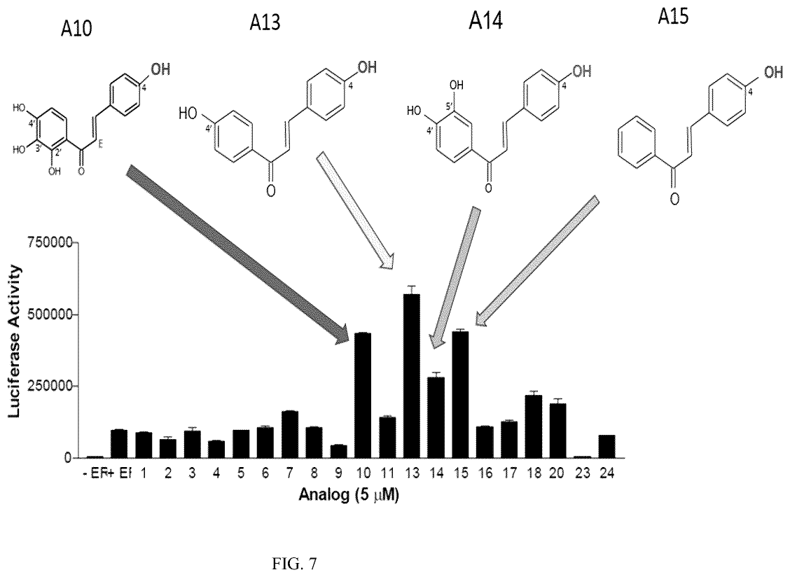

Luciferase Activity in NKG2E-TK-Luc-Transfected U2OS-ERβ Cells with Doxycycline. Doxycycline-dependent U2OS cells were transfected with NKG2E-TK-Luk and ERβ in the presence of E2 alone or in combination with each of the compounds of Tables 1A, 1B, and 1C. Luciferase activity for each of the compounds was recorded. See FIG. 7. As can be seen in FIGS. 7, A10, A13, A14, and A15 showed exceptional ERβ activity.

Example 7

Synthesis of Compounds of Tables 1A, 1B, and 1C. Compounds set forth in Tables 1A, 1B, and 1C were prepared per the synthetic procedures set forth below.

### A1

Synthesis of (E)-3-(4-fluorophenyl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH: H₂O (8 mL, V_{EtOH}:V_{H2O}=3:1) was added 4-fluorobenzaldehyde a (354 mg, 2.8 mmol) and KOH (1600 mg, 28 mmol). The result solution was stirred for 12 h at 0° C. ˜ rt. The mixture was filtered off to give (E)-3-(4-fluorophenyl)-1-(2,3,4-trimethoxyphenyl)prop-2-en-1-one 2 (630 mg, 80%) as a white solid.

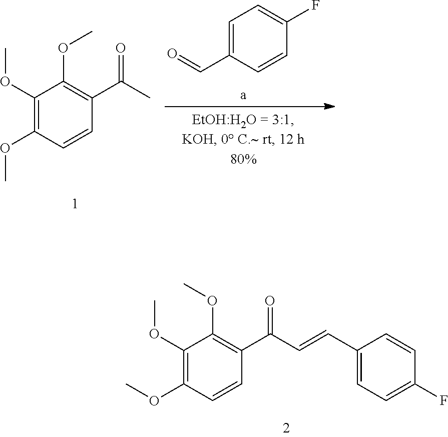 [SMILES: COc1ccc(C(C)=O)c(OC)c1OC ; O=Cc1ccc(F)cc1 ; COc1ccc(C(=O)/C=C/c2ccc(F)cc2)c(OC)c1OC]

Synthesis of (E)-3-(4-fluorophenyl)-1-(2,3,4-trihydroxyphenyl)prop-2-en-1-one A1. To a solution of (E)-3-(4-fluorophenyl)-1-(2,3,4-trimethoxyphenyl)prop-2-en-1-one 2 (200 mg, 0.63 mmol) in dry DCM (5 mL) was added dropwise BBr₃ (0.95 g, 3.8 mmol) at 0° C. under N₂. The resulting mixture was stirred at temperature for 12 h, and then quenched by water at temperature. Finally, take crude product and was purified by flash reversed phase column (MeOH in water=5% to 100%, 60 min) to give (E)-3-(4-fluorophenyl)-1-(2,3,4-trihydroxyphenyl)prop-2-en-1-one A1 (63 mg, 36%) as a yellow solid. ¹H NMR (400 MHZ, DMSO-d₆) δ 13.35 (s, 1H), 10.16 (s, 1H), 8.64 (s, 1H), 8.05-7.88 (m, 3H), 7.85-7.63 (m, 2H), 7.31 (t, J=8.8 Hz, 2H), 6.45 (d, J=8.9 Hz, 1H).

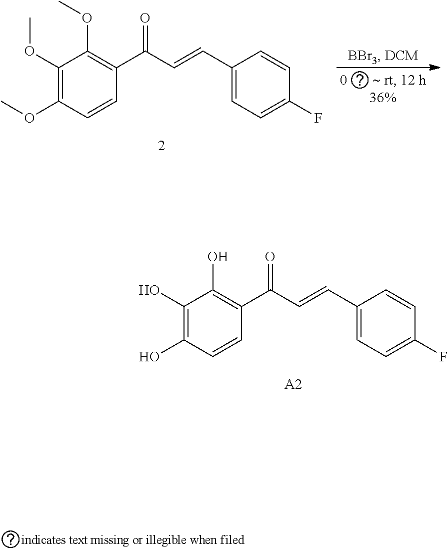 [SMILES: COc1ccc(C(=O)/C=C/c2ccc(F)cc2)c(OC)c1OC ; O=C(/C=C/c1ccc(F)cc1)c1ccc(O)c(O)c1O ; "A2"]

### A2

Synthesis of (E)-3-(3-fluorophenyl)-1-(2,3,4-trimethoxyphenyl) prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added 3-fluorobenzaldehyde a (354 mg, 2.8 mmol) and KOH (1600 mg, 28 mmol). The result solution was stirred for 12 h at 0° C.˜rt. The mixture was filtered off to give (E)-3-(3-fluorophenyl)-1-(2,3,4-trimethoxyphenyl)prop-2-en-1-one 2 (630 mg, 80%) as a white solid.

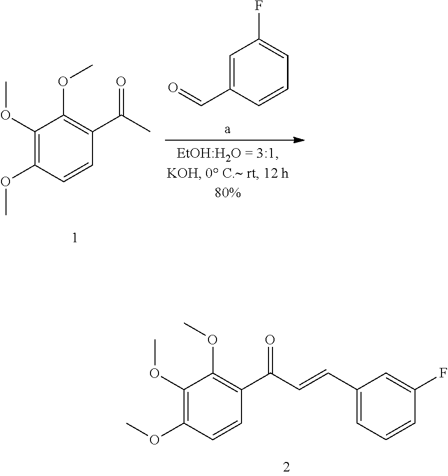 [SMILES: COc1ccc(C(C)=O)c(OC)c1OC ; O=Cc1cccc(F)c1 ; COc1ccc(C(=O)/C=C/c2cccc(F)c2)c(OC)c1OC]

Synthesis of (E)-3-(3-fluorophenyl)-1-(2,3,4-trihydroxyphenyl)prop-2-en-1-one A2. To a solution of (E)-3-(3-fluorophenyl)-1-(2,3,4-trimethoxyphenyl)prop-2-en-1-one 2 (500 mg, 1.58 mmol) in dry DCM (5 mL) was added dropwise BBr₃ (2 g, 7.9 mmol) at 0° C. under N₂. The resulting mixture was stirred at temperature for 12 h, and then quenched by water at temperature. Finally crude product was purified by flash reversed phase column (MeOH in water=5% to 100%, 60 min) to give (E)-3-(3-fluorophenyl)-1-(2,3,4-trihydroxyphenyl)prop-2-en-1-one A2 (65 mg, 15%) as a yellow solid. ¹H NMR (400 MHZ, DMSO-d₆) δ 13.31 (s, 1H), 10.25 (s, 1H), 8.70 (s, 1H), 8.03 (d, J=15.5 Hz, 1H), 7.88 (dt, J=10.1, 2.1 Hz, 1H), 7.83-7.74 (m, 2H), 7.69 (s, 1H), 7.51 (td, J=8.0, 6.1 Hz, 1H), 7.30 (td, J=8.7, 2.6 Hz, 1H), 6.46 (d, J=8.9 Hz, 1H).

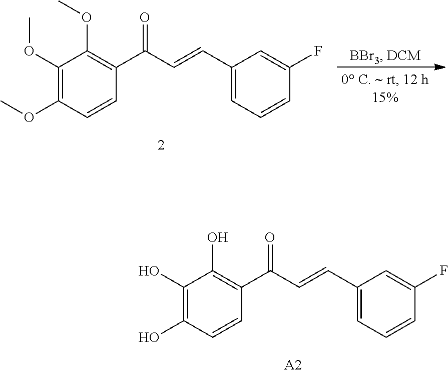 [SMILES: COc1ccc(C(=O)/C=C/c2cccc(F)c2)c(OC)c1OC ; O=C(/C=C/c1cccc(F)c1)c1ccc(O)c(O)c1O ; "A2"]

### A3

Synthesis of (E)-3-(2-fluorophenyl)-1-(2,3,4-trimethoxyphenyl)prop-2-en-1-one 2. To a solution of 1-(2,3,4-trimethoxyphenyl) ethan-1-one 1 (600 mg, 2.8 mmol) in EtOH:H₂O (8 mL, V_{EtOH}:V_{H2O}=3:1) was added 2-fluorobenzaldehyde a (354 mg, 2.8 mmol) and KOH (1600 mg, 28 mmol). The result solution was stirred for 12 h at 0° C.˜rt. The mixture was filtered off to give (E)-3-(2-fluorophenyl)-1-(2,3,4-trimethoxyphenyl)prop-2-en-1-one 2 (630 mg, 80%) as a white solid.

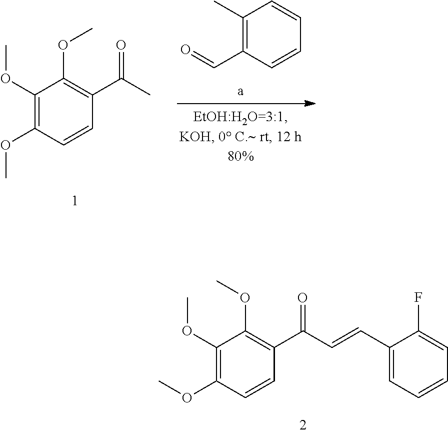

Synthesis of (E)-3-(2-fluorophenyl)-1-(2,3,4-trihydroxyphenyl)prop-2-en-1-one. To a solution of (E)-3-(2-fluorophenyl)-1-(2,3,4-trimethoxyphenyl)prop-2-en-1-one 2 (300 mg, 0.95 mmol) in dry DCM (5 mL) was added dropwise BBr₃ (1.2 g, 4.74 mmol) at 0° C. under N₂. The resulting mixture was stirred at temperature for 12 h, and then quenched by water at temperature. Finally, take crude product and was purified by flash reversed phase column (MeOH in water=5% to 100%, 60 min) to give (E)-3-(2-fluorophenyl)-1-(2,3,4-trihydroxyphenyl)prop-2-en-1-one A3 (235 mg, 90%) as a yellow solid. ¹H NMR (400 MHZ, DMSO-d₆) δ 13.27 (d, J=32.2 Hz, 1H), 10.27 (d, J=9.0 Hz, 1H), 8.72 (d, J=9.9 Hz, 1H), 8.15 (t, J=7.2 Hz, 1H), 8.03 (dd, J=15.5, 5.1 Hz, 1H), 7.90 (d, J=15.7 Hz, 1H), 7.82-7.75 (m, 1H), 7.74-7.68 (m, 1H), 7.58-7.44 (m, 1H), 7.41-7.24 (m, 2H), 6.46 (dd, J=8.9, 3.7 Hz, 1H).

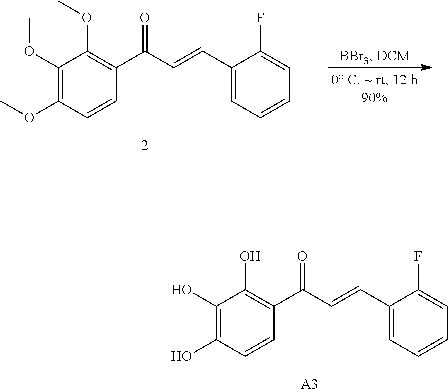 [SMILES: COc1ccc(C(=O)/C=C/c2ccccc2F)c(OC)c1OC ; O=C(/C=C/c1ccccc1F)c1ccc(O)c(O)c1O ; "A3"]

Synthesis of (E)-3-(m-tolyl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added 3-methylbenzaldehyde a (354 mg, 2.8 mmol) and KOH (1600 mg, 28 mmol). The result solution was stirred for 12 h at 0° C.˜rt. The mixture was filtered off to give (E)-3-(m-tolyl)-1-(2,3,4-trimethoxyphenyl)prop-2-en-1-one 2 (630 mg, 80%) as a white solid.

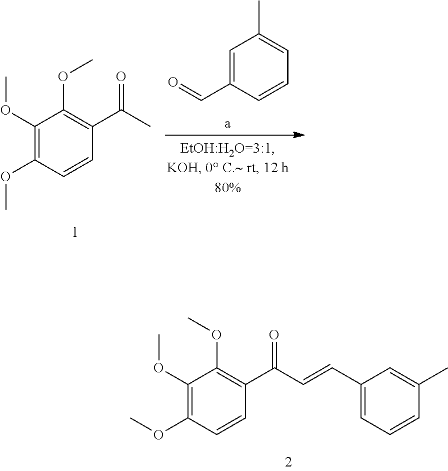

Synthesis of (E)-3-(m-tolyl)-1-(2,3,4-trihydroxyphenyl)prop-2-en-1-one A4. To a solution of (E)-3-(m-tolyl)-1-(2,3,4-trimethoxyphenyl)prop-2-en-1-one 2 (300 mg, 0.95 mmol) in dry DCM (5 mL) was added dropwise BBr₃ (1.2 g, 4.74 mmol) at 0° C. under N₂. The resulting mixture was stirred at temperature for 12 h, and then quenched by water at temperature. Finally, crude product was purified by flash reversed phase column (MeOH in water=5% to 100%, 60 min) to give (E)-3-(m-tolyl)-1-(2,3,4-trihydroxyphenyl)prop-2-en-1-one A4 (140 mg, 54%) as a yellow solid. ¹H NMR (400 MHZ, DMSO-d₆) δ 13.44 (s, 1H), 10.18 (s, 1H), 8.66 (s, 1H), 7.92 (d, J=15.5 Hz, 1H), 7.83-7.70 (m, 4H), 7.28 (d, J=8.0 Hz, 2H), 6.45 (d, J=8.9 Hz, 1H), 2.36 (s, 3H).

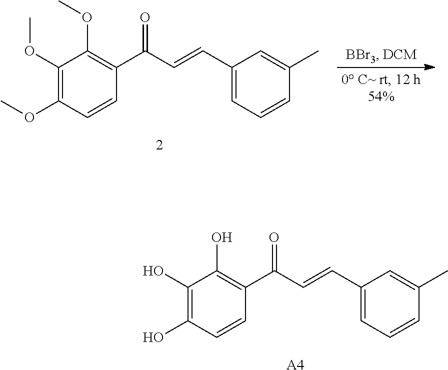

### A5

Synthesis of (E)-3-(m-tolyl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added 3-methylbenzaldehyde a (342 mg, 2.8 mmol) and KOH (1600 mg, 28 mmol). The result solution was stirred for 12 h at 0° C.˜rt. The mixture was filtered off to give (E)-3-(m-tolyl)-1-(2,3,4-trimethoxyphenyl)prop-2-en-1-one 2 (150 mg, 6.7%) as a white solid.

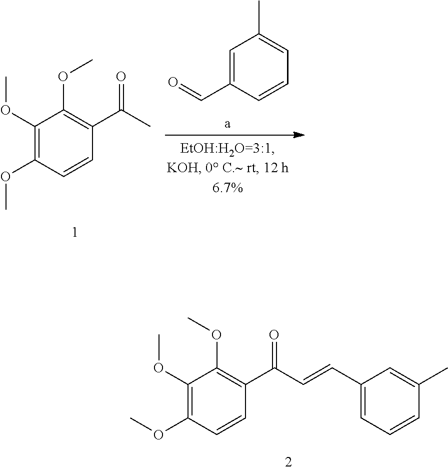

Synthesis of (E)-3-(m-tolyl)-1-(2,3,4-trihydroxyphenyl)prop-2-en-1-one A5. To a solution of (E)-3-(m-tolyl)-1-(2,3,4-trimethoxyphenyl)prop-2-en-1-one 2 (150 mg, 0.54 mmol) in dry DCM (5 mL) was added dropwise BBr₃ (684 mg, 2.73 mmol) at 0° C. under N₂. The resulting mixture was stirred at temperature for 12 h, and then quenched by water at temperature. Finally, crude product was purified by flash reversed phase column (MeOH in water=5% to 100%, 60 min) to give (E)-3-(m-tolyl)-1-(2,3,4-trihydroxyphenyl)prop-2-en-1-one A5 (80 mg, 55%) as a yellow solid. ¹H NMR (400 MHZ, DMSO-d₆) δ 13.41 (s, 1H), 10.22 (s, 1H), 8.69 (s, 1H), 7.93 (s, 1H), 7.83-7.71 (m, 3H), 7.67 (d, J=7.7 Hz, 1H), 7.36 (s, 1H), 7.29 (s, 1H), 6.46 (d, J=8.9 Hz, 1H), 2.37 (s, 3H).

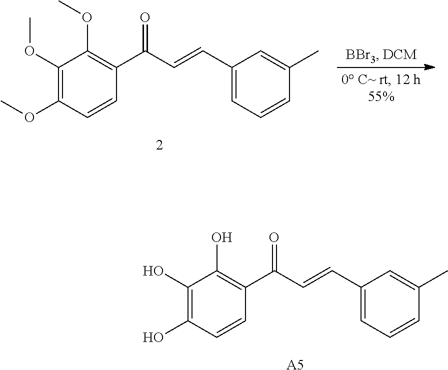

### A6

Synthesis of E)-3-(o-tolyl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added 2-methylbenzaldehyde a (354 mg, 2.8 mmol) and KOH (1600 mg, 28 mmol). The result solution was stirred for 12 h at 0° C.˜rt. The mixture was filtered off to give (E)-3-(o-tolyl)-1-(2,3,4-trimethoxyphenyl)prop-2-en-1-one 2 (630 mg, 80%) as a white solid.

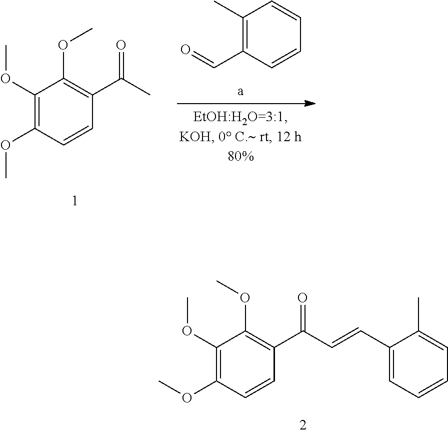

Synthesis of (E)-3-(o-tolyl)-1-(2,3,4-trihydroxyphenyl)prop-2-en-1-one A6. To a solution of (E)-3-(o-tolyl)-1-(2,3,4-trimethoxyphenyl)prop-2-en-1-one 2 (300 mg, 0.95 mmol) in dry DCM (5 mL) was added dropwise BBr₃ (1.2 g, 4.74 mmol) at 0° C. under N₂. The resulting mixture was stirred at temperature for 12 h, and then quenched by water at temperature. Finally, crude product was purified by flash reversed phase column (MeOH in water=5% to 100%, 60 min) to give (E)-3-(o-tolyl)-1-(2,3,4-trihydroxyphenyl)prop-2-en-1-one A6 (150 mg, 95%) as a yellow solid. ¹H NMR (400 MHZ, DMSO-d₆) δ 13.37 (s, 1H), 10.23 (s, 1H), 8.70 (s, 1H), 8.12-7.95 (m, 2H), 7.86 (d, J=15.4 Hz, IH), 7.75 (d, J=9.0 Hz, 1H), 7.40-7.21 (m, 3H), 6.45 (d, J=8.9 Hz, 1H), 2.46 (s, 3H).

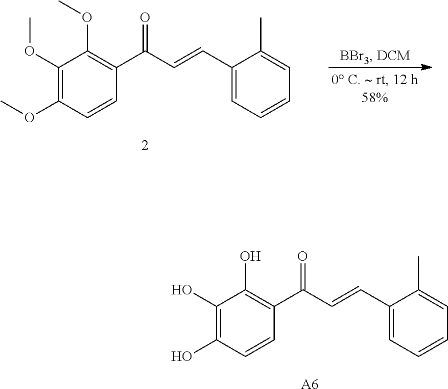 [SMILES: COc1ccc(C(=O)/C=C/c2ccccc2C)c(OC)c1OC ; Cc1ccccc1/C=C/C(=O)c1ccc(O)c(O)c1O ; "A6"]

### A7

Synthesis of (E)-3-(pyridin-4-yl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added isonicotinaldehyde (305 mg, 2.8 mmol) and KOH (156 mg, 2.8 mmol). The result solution was stirred for 5 min at rt. The mixture was concentrated and purified by RP-column to give (E)-3-(pyridin-4-yl)-1-(2,3,4-trimethoxyphenyl)prop-2-en-1-one 2 (510 mg, 59%) as a yellow solid.

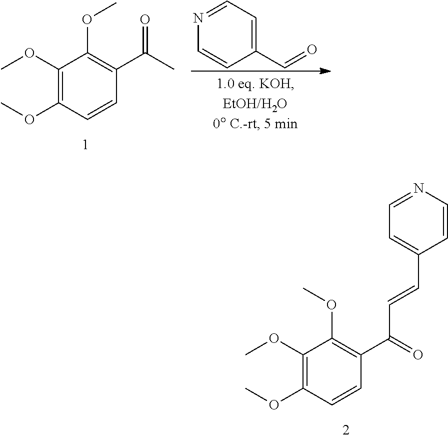

Synthesis of (E)-3-(pyridin-4-yl)-1-(2,3,4-trihydroxyphenyl)prop-2-en-1-one A7. To a solution of 2 (200 mg, 0.67 mmol) in dry DCM (5 mL) was added dropwise BBr₃ (0.95 g, 3.8 mmol) at 0° C. under N₂. The resulting mixture was stirred at room temperature for 6 h, and then quenched by water. The organic layer was concentrated and purified by RP-column to give a crude. The crude was further purified by Prep-HPLC to give (E)-3-(pyridin-4-yl)-1-(2,3,4-trihydroxyphenyl)prop-2-en-1-one A7 (35 mg, 20%) as a yellow solid. ¹H NMR (400 MHZ, DMSO-d₆) δ 13.14 (s, 1H), 8.72-8.58 (m, 2H), 8.19 (d, J=15.6 Hz, 1H), 7.89-7.81 (m, 2H), 7.82-7.68 (m, 2H), 6.47 (d, J=8.9 Hz, 1H).

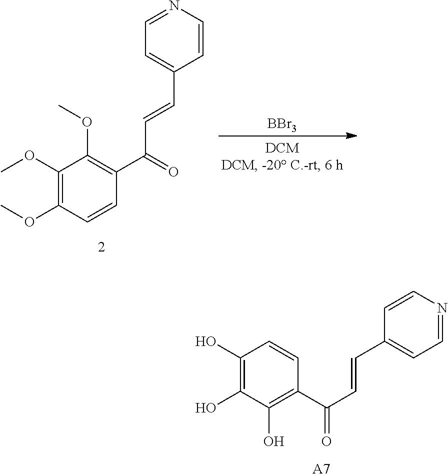

### A8

Synthesis of (E)-3-(pyridin-3-yl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added nicotinaldehyde (305 mg, 2.8 mmol) and KOH (156 mg, 28 mmol). The result solution was stirred for 5 min at rt. The mixture was concentrated and purified by RP-column to give (E)-3-(pyridin-3-yl)-1-(2,3,4-trimethoxyphenyl)prop-2-en-1-one 2 (520 mg, 60%) as a yellow solid.

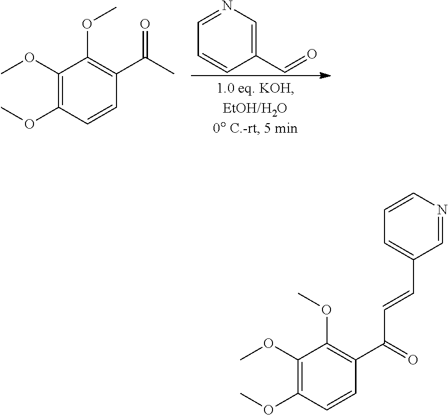

Synthesis of (E)-3-(pyridin-3-yl)-1-(2,3,4-trihydroxyphenyl)prop-2-en-1-one A8. To a solution of 2 (200 mg, 0.67 mmol) in dry DCM (5 mL) was added dropwise BBr₃ (0.95 g, 3.8 mmol) at 0° C. under N₂. The resulting mixture was stirred at room temperature for 6 h, and then quenched by water. The organic layer was concentrated and purified by RP-column to give a crude. The crude was further purified by Prep-HPLC to give (E)-3-(pyridin-3-yl)-1-(2,3,4-trihydroxyphenyl)prop-2-en-1-one A8 (15 mg, 8.7%) as a yellow solid. ¹H NMR (400 MHZ, DMSO-d₆) δ 13.28 (s, 1H), 9.03 (d, J=2.1 Hz, 1H), 8.62 (dd, J=4.7, 1.5 Hz, 1H), 8.37 (dt, J=8.1, 2.2 Hz, 1H), 8.11 (d, J=15.6 Hz, 1H), 7.92-7.66 (m, 2H), 7.50 (dd, J=8.0, 4.7 Hz, 1H), 6.46 (d, J=8.9 Hz, 1H).

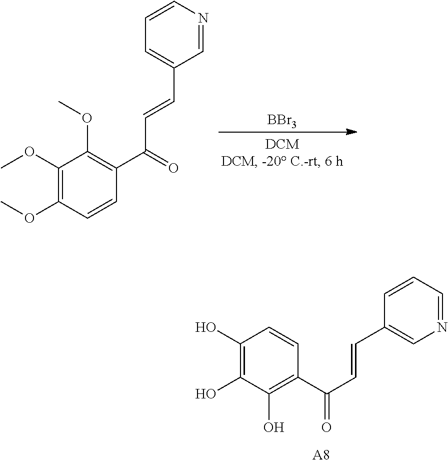

### A9

Synthesis of (E)-3-(pyridin-2-yl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added picolinaldehyde (305 mg, 2.8 mmol) and KOH (156 mg, 28 mmol). The result solution was stirred for 5 min at rt. The mixture was concentrated and purified by RP- column to give (E)-3-(pyridin-2-yl)-1-(2,3,4-trimethoxyphenyl)prop-2-en-1-one 2 (500 mg, 58%) as a yellow solid.

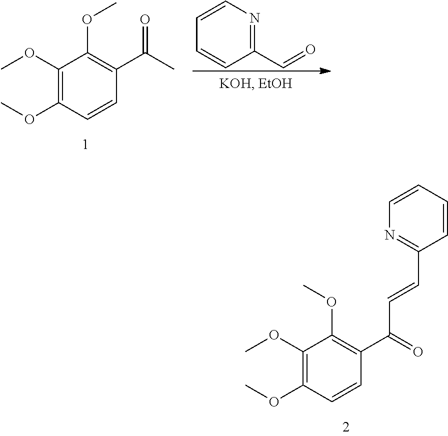

Synthesis of (E)-3-(pyridin-2-yl)-1-(2,3,4-trihydroxyphenyl)prop-2-en-1-one A9. To a solution of 2 (200 mg, 0.67 mmol) in dry DCM (5 mL) was added dropwise BBr₃ (0.95 g, 3.8 mmol) at 0° C. under N₂. The resulting mixture was stirred at room temperature for 6 h, and then quenched by water. The organic layer was concentrated and purified by RP-column to give a crude. The crude was further purified by Prep-HPLC to give (E)-3-(pyridin-2-yl)-1-(2,3,4-trihydroxyphenyl)prop-2-en-1-one A9 (30 mg, 17%) as a yellow solid. ¹H NMR (400 MHZ, DMSO-d₆) δ 13.15 (s, 1H), 10.31 (s, 1H), 8.80-8.64 (m, 2H), 8.19 (d, J=15.3 Hz, 1H), 7.98-7.86 (m, 2H), 7.78 (d, J=15.3 Hz, 1H), 7.61 (d, J=9.0 Hz, 1H), 7.45 (ddd, J=6.8, 4.7, 1.9 Hz, 1H), 6.48 (d, J=8.9 Hz, 1H).

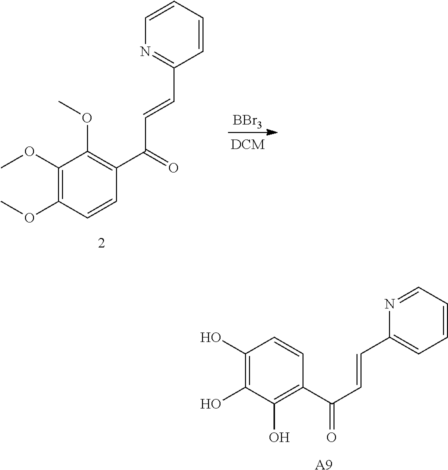

### A10

Synthesis of (E)-3-(3-methoxyphenyl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added 3-methoxybenzaldehyde a (387 mg, 2.8 mmol) and KOH (1600 mg, 28 mmol). The result solution was stirred for 12 h at 0° C.˜rt. The mixture was filtered off to give (E)-3-(3-methoxyphenyl)-1-(2,3,4-trimethoxyphenyl)prop-2-en-1-one 2 (600 mg, 64%) as a white solid.

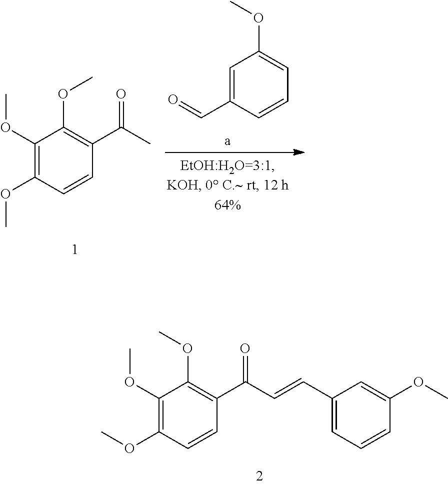

Synthesis of (E)-3-(3-hydroxyphenyl)-1-(2,3,4-trihydroxyphenyl)prop-2-en-1-one A10. To a solution of (E)-3-(3-methoxyphenyl)-1-(2,3,4-trimethoxyphenyl)prop-2-en-1-one 2 (200 mg, 0.61 mmol) in dry DCM (5 mL) was added dropwise BBr₃ (762 mg, 3 mmol) at 0° C. under N₂. The resulting mixture was stirred at temperature for 12 h, and then quenched by water at temperature. Finally crude product was purified by flash reversed phase column (MeOH in water=5% to 100%, 60 min) to give (E)-3-(3-hydroxyphenyl)-1-(2,3,4-trihydroxyphenyl)prop-2-en-1-one A10 (63 mg, 38%) as a yellow solid. ¹H NMR (400 MHZ, DMSO-d₆) δ 13.60 (s, 1H), 10.12 (s, 2H), 8.63 (s, 1H), 7.73 (q, J=9.0, 8.5 Hz, 5H), 6.84 (d, J=8.6 Hz, 2H), 6.43 (d, J=8.9 Hz, 1H).

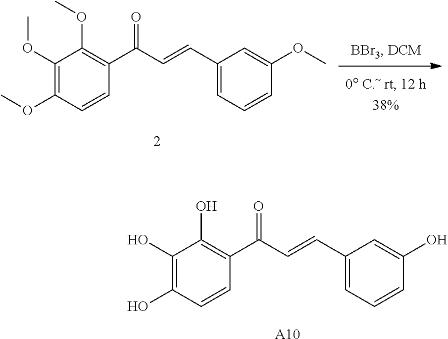

### A11

Synthesis of (E)-3-(4-aminophenyl)-1-(2,3,4-trimethoxyphenyl)prop-2-en-1-one 2. To a solution of 1-(2,3,4-trimethoxyphenyl)ethan-1-one 1 (600 mg, 2.8 mmol) in EtOH:H₂O (8 mL, V_{EtOH}:V_{H2O}=3:1) was added 4-aminobenzaldehyde (338 mg, 2.8 mmol) and KOH (156 mg, 28 mmol). The result solution was stirred for 24 h at rt. The mixture was concentrated and purified by FCC to give (E)-3-(4-aminophenyl)-1-(2,3,4-trimethoxyphenyl)prop-2-en-1-one 2 (300 mg, 34%) as a yellow solid.

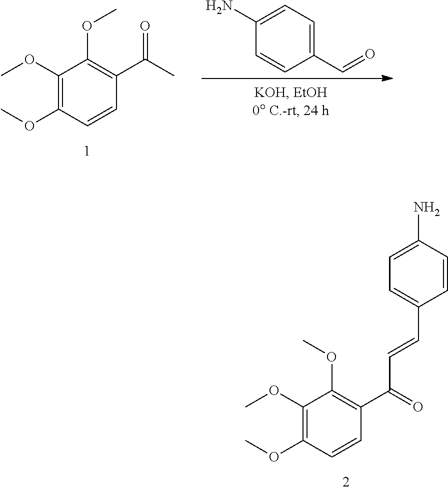 [SMILES: COc1ccc(C(C)=O)c(OC)c1OC ; Nc1ccc(C=O)cc1 ; COc1ccc(C(=O)/C=C/c2ccc(N)cc2)c(OC)c1OC]

Synthesis of (E)-3-(4-aminophenyl)-1-(2,3,4-trihydroxyphenyl)prop-2-en-1-one A11. To a solution of 2 (200 mg, 0.64 mmol) in dry DCM (5 mL) was added dropwise BBr₃ (0.95 g, 3.8 mmol) at 0° C. under N₂. The resulting mixture was stirred at room temperature for 6 h, and then quenched by water. The organic layer was concentrated and purified by RP-column to give a crude. The crude was further purified by Prep-HPLC to give (E)-3-(4-aminophenyl)-1-(2,3,4-trihydroxyphenyl)prop-2-en-1-one A11 (30 mg, 17%) as a yellow solid. ¹H NMR (400 MHZ, DMSO-d₆) δ 13.86 (s, 1H), 9.99 (s, 1H), 8.55 (s, 1H), 7.76-7.62 (m, 2H), 7.62-7.52 (m, 3H), 6.59 (d, J=8.2 Hz, 2H), 6.40 (d, J=8.8 Hz, 1H), 5.95 (s, 2H).

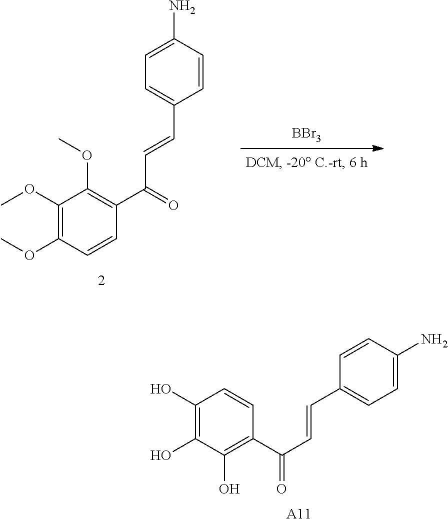

### A18

Synthesis of 4-((tetrahydro-2H-pyran-2-yl)oxy)benzaldehyde 2. A solution of 3,4-dihydro-2H-pyran (4.1 g 49 mmol) in dichloromethane (30 mL) was added dropwise into a well-stirred suspension of 4-hydroxybenzaldehyde (2 g, 16 mmol) and pyridinium p-toluenesulfonate (160 mg, 0.6 mmol) in dichloromethane (5 mL). The mixture was stirred at room temperature overnight, then extracted with brine and dried over MgSO₄. The solvent was removed under reduced pressure and the crude compound was purified by column chromatography on silica gel using an elution of 20% EtOAc/hexane to give 2 as a yellow oil (2 g, 60%).

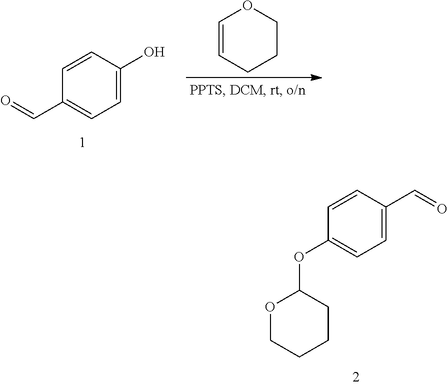 [SMILES: O=Cc1ccc(O)cc1 ; C1=COCCC1 ; O=Cc1ccc(OC2CCCCO2)cc1]

Synthesis of (E)-1-(pyridin-2-yl)-3-(4-((tetrahydro-2H-pyran-2-yl)oxy)phenyl) prop-2-en-1-one 3. To a solution of 2 (500 mg, 2.4 mmol) in EtOH (8 mL) was added 1-(pyridin-4-yl) ethan-1-one (293 mg, 2.4 mmol) and KOH (134 mg, 2.4 mmol). The result solution was stirred for 5 min at rt. The mixture was concentrated and purified by RP-column to give a crude. The crude was further purified by Prep-HPLC to give (E)-1-(pyridin-2-yl)-3-(4-((tetrahydro-2H-pyran-2-yl)oxy)phenyl)prop-2-en-1-one 3 (150 mg, 20%) as a yellow solid.

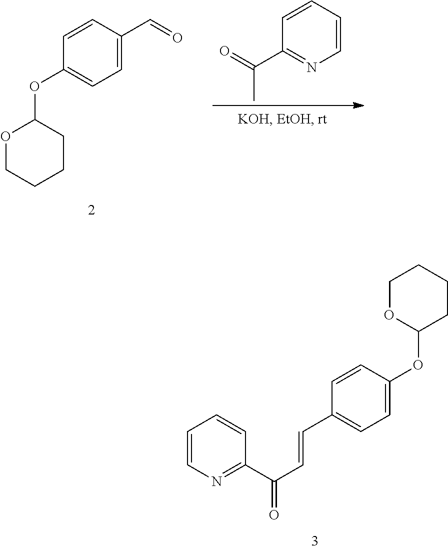

Synthesis of (E)-3-(4-hydroxyphenyl)-1-(pyridin-2-yl)prop-2-en-1-one. To a solution of 3 (200 mg, 0.64 mmol) in MeOH (1 mL) was added PTSA (11.0 mg, 0.06 mmol). The resulting mixture was stirred at room temperature for 5 h. The mixture was concentrated and purified by RP-column to give (E)-3-(4-hydroxyphenyl)-1-(pyridin-2-yl)prop-2-en-1-one A18 (6 mg, 8.3%) as a yellow solid. ¹H NMR (400 MHZ, DMSO-d₆) δ 10.25 (s, 1H), 8.79 (dt, J=4.7, 1.3 Hz, 1H), 8.14-8.00 (m, 3H), 7.79 (d, J=16.0 Hz, 1H), 7.74-7.62 (m, 3H), 7.05-6.73 (m, 2H).

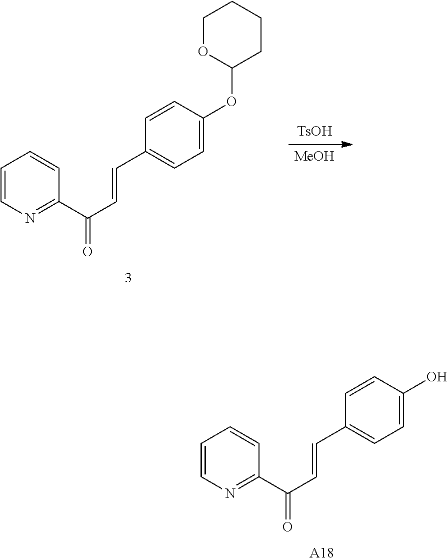

### A13

Synthesis of (E)-1,3-bis(4-methoxyphenyl)prop-2-en-1-one 2. To a solution of 4-methoxybenzaldehyde 1 (300 mg, 2.2 mmol) in EtOH:H₂O (4 mL, V_{EtOH}:V_{H2O}=3:1) was added 1-(4-methoxyphenyl)ethan-1-one a (330 mg, 2.2 mmol) and KOH (246 mg, 4.4 mmol). The result solution was stirred for 12 h at 0° C.˜rt. The mixture was filtered off to give (E)-1,3-bis(4-methoxyphenyl)prop-2-en-1-one 2 (0.57 g, 96%) as a white solid.

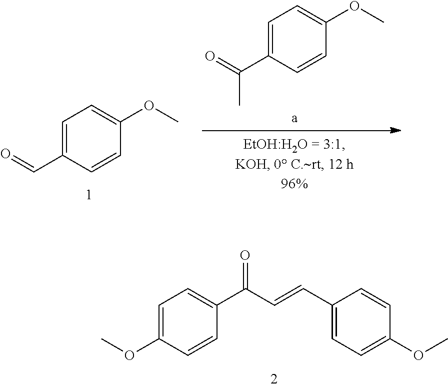

Synthesis of (E)-1,3-bis(4-hydroxyphenyl)prop-2-en-1-one. To a solution of (E)-1,3-bis(4-methoxyphenyl)prop-2-en-1-one 2 (200 mg, 0.74 mmol) in dry DCM (5 mL) was added dropwise BBr₃ (0.93 g, 3.73 mmol) at 0° C. under N₂. The resulting mixture was stirred at temperature for 12 h, and then quenched by water at temperature. Finally crude product was purified by flash reversed phase column (MeOH in water=5% to 100%, 60 min) to give N-benzyl-2,3,4-trihydroxybenzamide A13 (150 mg, 83%) as a yellow solid. ¹H NMR (400 MHZ, DMSO-d₆) δ 10.30 (s, 1H), 8.03 (d, J=8.7 Hz, 2H), 7.76-7.53 (m, 4H), 6.85 (dd, J=19.7, 8.6 Hz, 4H).

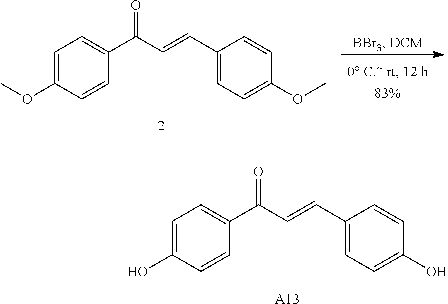

### A14

Synthesis of (E)-1-(3,4-dimethoxyphenyl)-3-(4-methoxyphenyl)prop-2-en-1-one 2. To a solution of 4-methoxybenzaldehyde 1 (300 mg, 2.2 mmol) in EtOH:H₂O (4 mL, V_{EtOH}:V_{H2O}=3:1) was added 1-(3,4-dimethoxyphenyl)ethan-1-one a (397 mg, 2.2 mmol) and KOH (246 mg, 4.4 mmol). The result solution was stirred for 12 h at 0° C.˜rt. The mixture was filtered off to give (E)-1-(3,4-dimethoxyphenyl)-3-(4-methoxyphenyl)prop-2-en-1-one 2 (0.64 g, 97%) as a white solid.

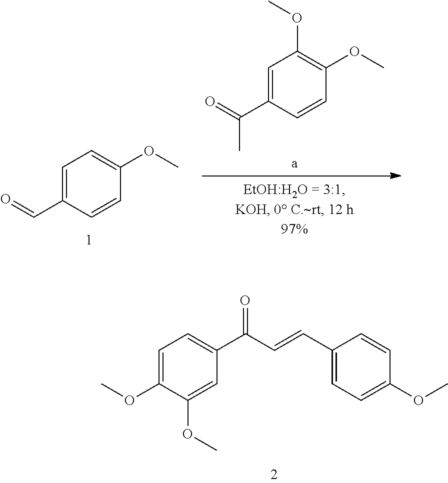

Synthesis of (E)-1-(3,4-dihydroxyphenyl)-3-(4-hydroxyphenyl)prop-2-en-1-one A14. To a solution of (E)-1-(3,4-dimethoxyphenyl)-3-(4-methoxyphenyl)prop-2-en-1-one 2 (300 mg, 1 mmol) in dry DCM (5 mL) was added dropwise BBr₃ (1.25 g, 5 mmol) at 0° C. under N₂. The resulting mixture was stirred at temperature for 12 h, and then quenched by water at temperature. Finally crude product was purified by flash reversed phase column (MeOH in water=5% to 100%, 60 min) to give (E)-1-(3,4-dihydroxyphenyl)-3-(4-hydroxyphenyl)prop-2 A14 (115 mg, 45%) as a yellow solid. ¹H NMR (400 MHZ, DMSO-d₆) δ 9.94 (s, 3H), 7.69 (d, J=8.6 Hz, 2H), 7.63-7.54 (m, 3H), 7.49 (d, J=2.0 Hz, 1H), 6.83 (t, J=8.8 Hz, 3H).

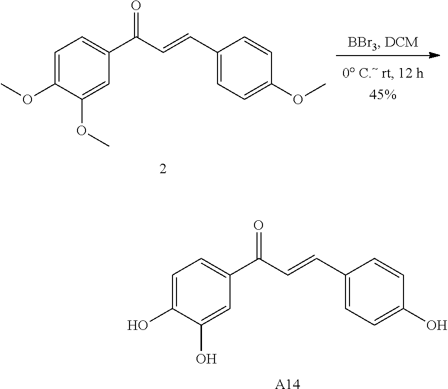

### A15

Synthesis of (E)-3-(4-hydroxyphenyl)-1-phenylprop-2-en-1-one A15. To a solution of 4-hydroxybenzaldehyde 1 (100 mg, 0.82 mmol) in EtOH (5 mL) was added acetophenone a (98 mg, 0.82 mmol) and KOH (92 mg, 1.64 mmol). The result solution was stirred for 12 h at room temperature. The mixture was diluted with EA (50 mL), washed with brine, concentrated under reduced pressure and purified by column chromatography on silica gel with PE/EA=5/1 to give (E)-3-(4-hydroxyphenyl)-1-phenylprop-2-en-1-one A15 (40 mg, 23%) as a yellow solid. ¹H NMR (400 MHZ, Chloroform-d) δ 8.06-7.97 (m, 2H), 7.78 (d, J=15.7 Hz, 1H), 7.62-7.54 (m, 3H), 7.51 (t, J=7.5 Hz, 2H), 7.41 (d, J=15.6 Hz, 1H), 6.89 (d, J=8.6 Hz, 2H).

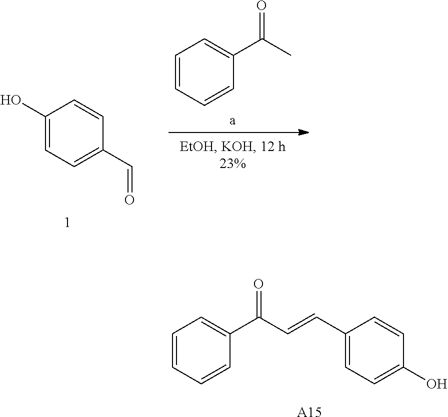

### A16

Synthesis of 4-((tetrahydro-2H-pyran-2-yl)oxy)benzaldehyde 2. A solution of 3,4-dihydro-2H-pyran (4.1 g 49 mmol) in dichloromethane (30 mL) was added dropwise into a well-stirred suspension of 4-hydroxybenzaldehyde (2 g, 16 mmol) and pyridinium p-toluenesulfonate (160 mg, 0.6 mmol) in dichloromethane (5 mL). The mixture was stirred at room temperature overnight, then extracted with brine and dried over MgSO₄. The solvent was removed under reduced pressure and the crude compound was purified by column chromatography on silica gel using an elution of 20% EtOAc/hexane to give 2 as a yellow oil (2 g, 60%).

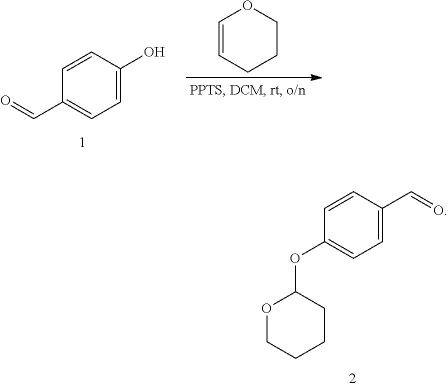 [SMILES: O=Cc1ccc(O)cc1 ; C1=COCCC1 ; O=Cc1ccc(OC2CCCCO2)cc1]

Synthesis of (E)-1-(pyridin-4-yl)-3-(4-((tetrahydro-2H-pyran-2-yl)oxy)phenyl)prop-2-en-1-one 2. To a solution of 2 (500 mg, 2.4 mmol) in EtOH (8 mL) was added 1-(pyridin-4-yl)ethan-1-one (293 mg, 2.4 mmol) and KOH (134 mg, 2.4 mmol). The result solution was stirred for 5 min at rt. The mixture was concentrated and purified by RP-column to give a crude. The crude was further purified by Prep-HPLC to give (E)-1-(pyridin-4-yl)-3-(4-((tetrahydro-2H-pyran-2-yl)oxy)phenyl)prop-2-en-1-one 3 (100 mg, 13%) as a yellow solid.

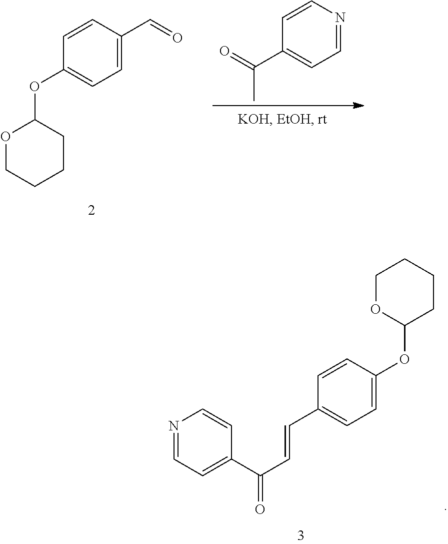

Synthesis of (E)-3-(4-hydroxyphenyl)-1-(pyridin-4-yl)prop-2-en-1-one. To a solution of 3 (100 mg, 0.32 mmol) in MeOH (1 mL) was added PTSA (5.5 mg, 0.03 mmol). The resulting mixture was stirred at room temperature for 5 h. The mixture was concentrated and purified by RP-column to give (E)-3-(4-hydroxyphenyl)-1-(pyridin-4-yl)prop-2-en-1-one A16 (60 mg, 83%) as a yellow solid. ¹H NMR (400 MHZ, DMSO-d₆) δ 10.20 (s, 1H), 8.91-8.66 (m, 2H), 8.02-7.91 (m, 2H), 7.82-7.61 (m, 4H), 6.93-6.76 (m, 2H).

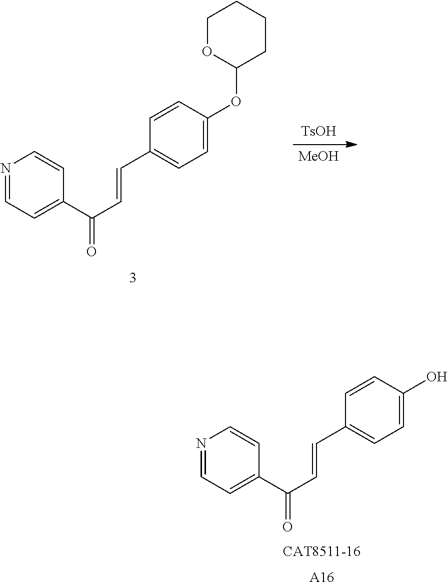

### A17

Synthesis of 4-((tetrahydro-2H-pyran-2-yl)oxy)benzaldehyde 2. A solution of 3,4-dihydro-2H-pyran (4.1 g 49 mmol) in dichloromethane (30 mL) was added dropwise into a well-stirred suspension of 4-hydroxybenzaldehyde (2 g, 16 mmol) and pyridinium p-toluenesulfonate (160 mg, 0.6 mmol) in dichloromethane (5 mL). The mixture was stirred at room temperature overnight, then extracted with brine and dried over MgSO₄. The solvent was removed under reduced pressure and the crude compound was purified by column chromatography on silica gel using an elution of 20% EtOAc/hexane to give 2 as a yellow oil (2 g, 60%).

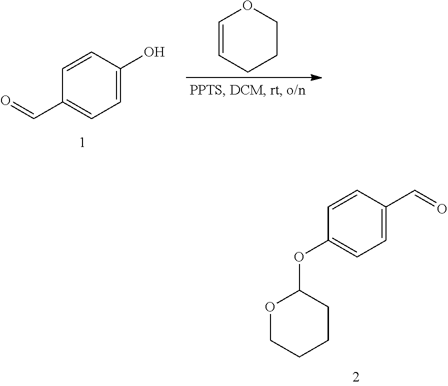 [SMILES: O=Cc1ccc(O)cc1 ; C1=COCCC1 ; O=Cc1ccc(OC2CCCCO2)cc1]

Synthesis of (E)-1-(pyridin-3-yl)-3-(4-((tetrahydro-2H-pyran-2-yl)oxy)phenyl)prop-2-en-1-one 2. To a solution of 2 (500 mg, 2.4 mmol) in EtOH (8 mL) was added 1-(pyridin-4-y1)ethan-1-one (293 mg, 2.4 mmol) and KOH (134 mg, 2.4 mmol). The result solution was stirred for 5 min at rt. The mixture was concentrated and purified by RP-column to give a crude. The crude was further purified by Prep-HPLC to give (E)-1-(pyridin-3-yl)-3-(4-((tetrahydro-2H-pyran-2-yl)oxy)phenyl)prop-2-en-1-one 3 (150 mg, 20%) as a yellow solid.

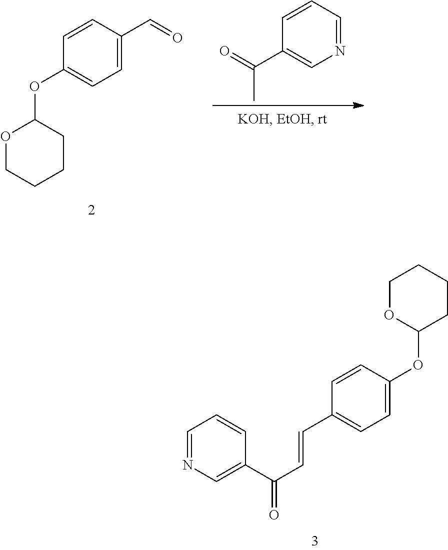

Synthesis of (E)-3-(4-hydroxyphenyl)-1-(pyridin-3-yl)prop-2-en-1-one. To a solution of 3 (100 mg, 0.32 mmol) in MeOH (1 mL) was added PTSA (5.5 mg, 0.03 mmol). The resulting mixture was stirred at room temperature for 5 h. The mixture was concentrated and purified by RP-column to give (E)-3-(4-hydroxyphenyl)-1-(pyridin-3-yl)prop-2-en-1-one A17 (30 mg, 41%) as a yellow solid. ¹H NMR (400 MHZ, DMSO-d₆) δ 10.31-9.99 (m, 1H), 9.30 (d, J=2.2 Hz, 1H), 8.81 (dd, J=4.8, 1.7 Hz, 1H), 8.44 (dt, J=8.0, 2.0 Hz, 1H), 7.82-7.69 (m, 4H), 7.60 (dd, J=8.0, 4.8 Hz, 1H), 6.93-6.79 (m, 2H).

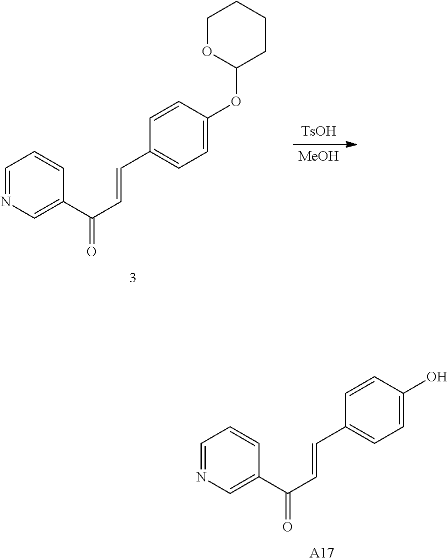

### A20

Synthesis of (E)-3-phenyl-1-(2,3,4-trimethoxyphenyl) prop-2-en-1-one 2. To a solution of 1-(2,3,4-trimethoxyphenyl)ethan-1-one 1 (1.0 g, 4.8 mmol) in EtOH:H₂O (10 mL, V_{EtOH}:V_{H2O}=3:1) was added benzaldehyde (508 mg, 4.8 mmol) and KOH (537 mg, 9.6 mmol). The result solution was stirred for 36 h at rt. The mixture was concentrated and purified by FCC to give (E)-3-phenyl-1-(2,3,4-trimethoxyphenyl)prop-2-en-1-one 2 (1.2 g, 85%) as a yellow solid.

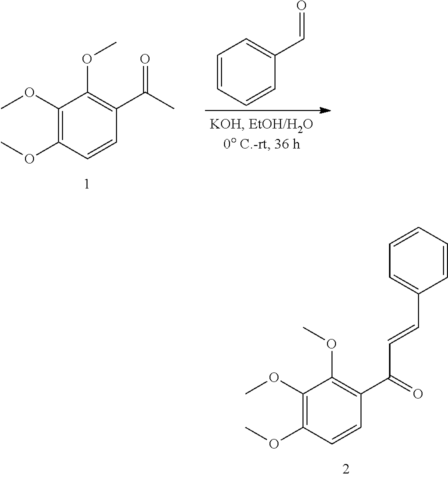 [SMILES: COc1ccc(C(C)=O)c(OC)c1OC ; O=Cc1ccccc1 ; COc1ccc(C(=O)/C=C/c2ccccc2)c(OC)c1OC]

Synthesis of 3-phenyl-1-(2,3,4-trimethoxyphenyl)propan-1-one 3. A solution of 2 (500 mg, 1 eq) in DCM (5 mL) was added 10% Pd/C (50 g). The mixture was stirred at room temperature for 6 hours under H₂ atmosphere. And then filtered through celite, the filtrate was concentrated under reduced pressure to give 3 as a crude (450 mg). The crude was used to the next step without purification.

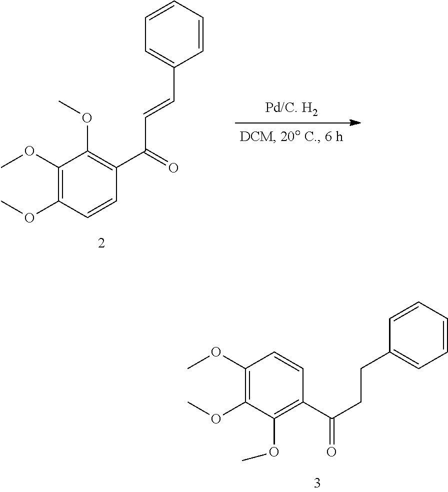 [SMILES: COc1ccc(C(=O)/C=C/c2ccccc2)c(OC)c1OC ; COc1ccc(C(=O)CCc2ccccc2)c(OC)c1OC]

Synthesis of 3-phenyl-1-(2,3,4-trihydroxyphenyl) propan-1-one. To a solution of 3 (300 mg, 1.0 mmol) in dry DCM (5 mL) was added dropwise BBr₃ (0.95 g, 3.8 mmol) at 0° C. under N₂. The resulting mixture was stirred at room temperature for 6 h, and then quenched by water. The organic layer was concentrated and purified by RP-column to give 3-phenyl-1-(2,3,4-trihydroxyphenyl)propan-1-one A20 (100 mg, 39%) as a yellow solid. ¹H NMR (400 MHZ, DMSO-d₆) δ 12.57 (s, 1H), 10.07 (s, 1H), 8.60 (s, 1H), 7.36 (d, J=8.9 Hz, 1H), 7.27 (d, J=4.4 Hz, 4H), 7.18 (q, J=4.4 Hz, 1H), 6.38 (d, J=8.9 Hz, 1H), 3.29 (t, J=7.6 Hz, 2H), 2.93 (t, J=7.6 Hz, 2H).

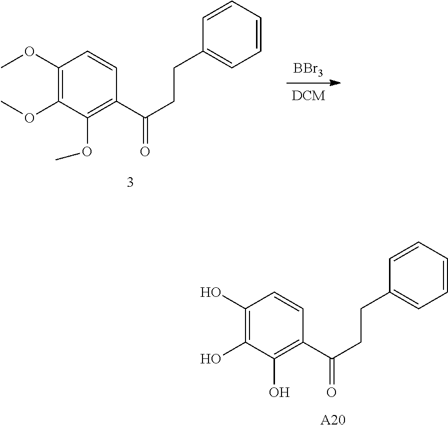

### A21

Synthesis of N-benzyl-2,3,4-trimethoxybenzamide 2. To a solution of 2,3,4-trimethoxybenzoic acid 1 (1.0 g, 4.7 mmol) in dry DMF (10 mL) was added phenylmethanamine a (0.6 g, 5.6 mmol), HOBt (0.75 g, 5.6 mmol) and EDCI (1.27 g, 6.6 mmol). The result solution was stirred for 2 h at 0° C. under N₂. The mixture was diluted with EA (50 mL), washed with brine, concentrated under reduced pressure and purified by column chromatography on silica gel with PE/EA=5/1 to give N-benzyl-2,3,4-trimethoxybenzamide 2 (2 g, 95%) as an oil.

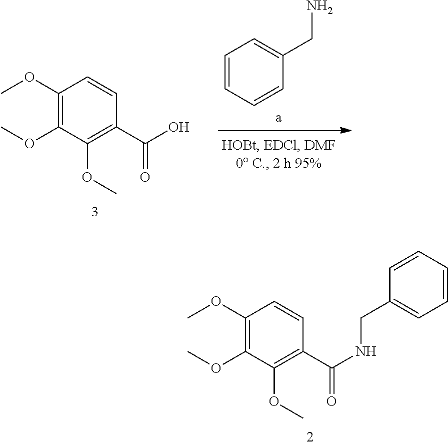

Synthesis of N-benzyl-2,3,4-trihydroxybenzamide. To a solution of N-benzyl-2,3,4-trimethoxybenzamide 2 (300 mg, 1 mmol) in dry DCM (5 mL) was added dropwise BBr₃ (1.25 g, 5 mmol) at 0° C. under N₂. The resulting mixture was stirred at temperature for 12 h, and then quenched by water at temperature. Finally, crude product was purified by flash reversed phase column (MeOH in water=5% to 100%, 60 min) to give N-benzyl-2,3,4-trihydroxybenzamide A21 (0.3 g, 70%) as a white solid. ¹H NMR (400 MHZ, DMSO-d₆) δ 12.96 (s, 1H), 9.50 (s, 1H), 9.10 (t, J=6.0 Hz, 1H), 8.43 (s, 1H), 7.45-7.15 (m, 6H), 6.31 (d, J=8.8 Hz, 1H), 4.47 (d, J=5.9 Hz, 2H).

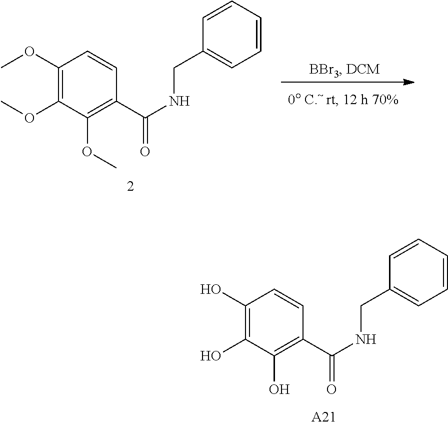

### A24

Synthesis of 5-(((2,3-dimethoxyphenyl)amino)methylene)-2,2-dimethyl-1,3-dioxane-4,6-dione 2. To a solution of dimethoxyaniline 1 (1.0 g, 6.5 mmol) in EtOH (10 mL) was added 2,2-dimethyl-1,3-dioxane-4,6-dione (0.94 g, 6.5 mmol). The result solution was stirred at 85° C. for 2.5 h. The mixture was filtered and washed by EtOH to give 5-(((2,3-dimethoxyphenyl)amino)methylene)-2,2-dimethyl-1,3-dioxane-4,6-dione 2 (1.5 g, 75%) as a yellow solid. The crude was used to the next step without purification.

Synthesis of 7,8-dimethoxyquinolin-4-ol 3. A solution of 2 (900 mg, 1 eq) in diphenyl ether (15 mL) was stirred at 260° C. for 1 h. The precipitate was filtered and washed with hexane. The brown solid was dried under vacuum to give 7,8-dimethoxyquinolin-4-ol 3 (540 mg, 90% yield).

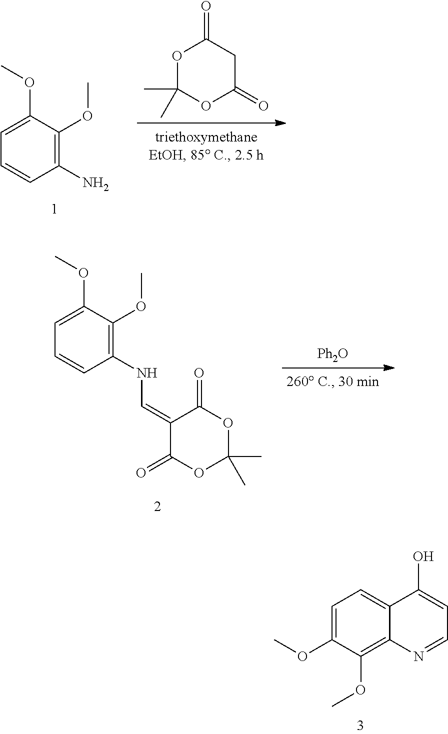

Synthesis of 4-chloro-7,8-dimethoxyquinoline 4. 3 (540 mg, 2.6 mmol) in phosphorus oxychloride (10 ml) and the reaction mixture was heated to 110° C. for 1 hours. The reaction mixture was cooled to room temperature and concentrated under reduced pressure. The crude product was diluted with water (20 mL) and the product was extracted with ethyl acetate (2×100 mL). The combined organic layer was dried over anhydrous sodium sulfate, filtered and concentrated under reduced pressure to yield 4-chloro-7,8-dimethoxyquinoline 4 (300 mg, 51% yield) as a yellow solid.

Synthesis of (E)-7,8-dimethoxy-4-styrylquinoline 5. A mixture of 4 (200 mg, 0.9 mmol), (E)-styrylboronic acid (132 mg, 0.9 mmol), K₂CO₃ (278 mg, 2.0 mmol), and Pd(dppf)Cl₂ (37 g, 0.05 mmol) in 1,4-dioxane (1 mL)/H₂O (0.3 mL) was stirred for 10 hours at 95° C. The mixture was cooled to room temperature, filtered through a pad of silica gel (EtOAc), and concentrated. The crude product was chromatographed on silica gel to afford (E)-7,8-dimethoxy-4-styrylquinoline 5 (130 g, 50%) as a yellow solid.

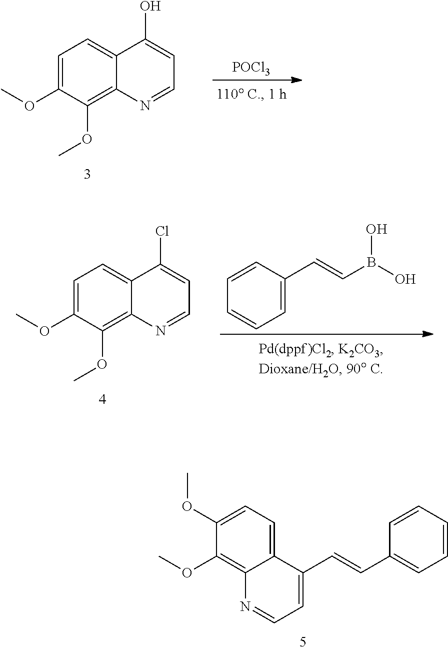

Synthesis of (E)-4-styrylquinoline-7,8-diol. To a solution of 5 (130 mg, 1.0 mmol) in dry DCM (5 mL) was added dropwise BBr₃ (0.95 g, 3.8 mmol) at 0° C. under N₂. The resulting mixture was stirred at room temperature for 6 h, and then quenched by water. The organic layer was concentrated and purified by RP-column to give (E)-4-styrylquinoline-7,8-diol A24 (40 mg, 34%) as a yellow solid. ¹H NMR (400 MHZ, DMSO-d₆) δ 9.29 (s, 1H), 8.74 (d, J=4.6 Hz, 1H), 8.14 (s, 1H), 7.99 (d, J=16.2 Hz, 1H), 7.82 (dd, J=8.4, 5.1 Hz, 3H), 7.67 (d, J=4.6 Hz, 1H), 7.56 (d, J=16.2 Hz, 1H), 7.44 (t, J=7.5 Hz, 2H), 7.37 (d, J=7.3 Hz, 1H), 7.24 (d, J=9.1 Hz, 1H).

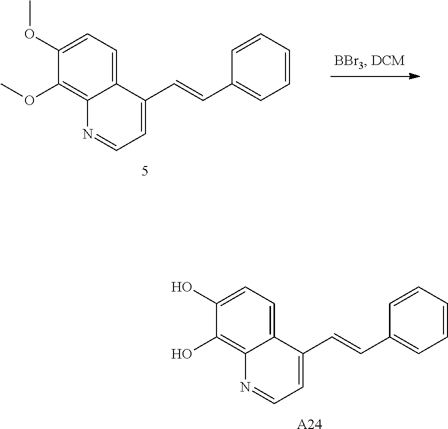

### Example 8

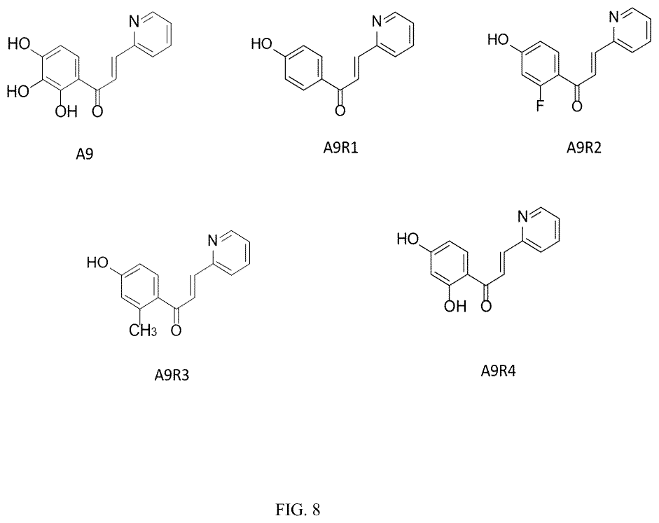

Synthesis of Analogs A9R1, A9R2, A9R3, and A9R4 (FIG. 8) were synthesized as follows:

A9R1: Synthesis of (E)-1-(4-hydroxyphenyl)-3-(pyridin-2-yl)prop-2-en-1-one. To a solution of 1-(4-hydroxyphenyl) ethan-1-one 1 (200 mg, 1.47 mmol) in EtOH (4 mL), picolinaldehyde 2 (157 mg, 1.47 mmol) and NaOH (588 mg, 14.7 mmol in 1 mL H₂O) were added. The reaction was stirred at RT for 2 h. The mixture was diluted with water and neutralized with 1M aqueous HCl solution (pH adjusted to 7). The precipitate was filtered and collected as the crude product. The crude product was purified by methanol recrystallization to give the title compound C9529-1 (29 mg, 9%) as a yellow solid. LCMS (ESI): m/z, 226 [M+H]+, RT=2.55 min. HPLC: 96%, RT=3.42 min. 1H NMR (400 MHZ, DMSO-d₆) δ 10.49 (s, 1H), 8.68 (dt, J=4.7, 1.4 Hz, 1H), 8.14 (d, J=15.4 Hz, 1H), 8.05-8.00 (m, 2H), 7.92-7.87 (m, 2H), 7.67 (d, J=15.4 Hz, 1H), 7.47-7.39 (m, 1H), 6.96-6.88 (m, 2H).

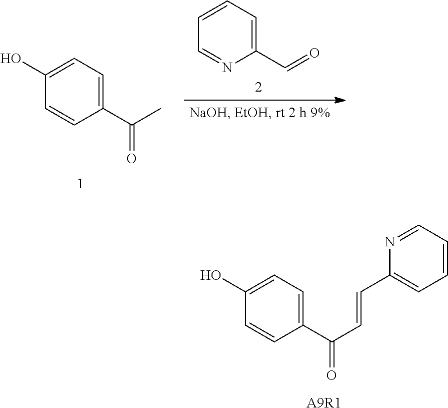

A9R2: Synthesis of (E)-1-(4-hydroxy-2-methylphenyl)-3-(pyridin-2-yl)prop-2-en-1-one. To a stirred solution of 1-(4-hydroxy-2-methylphenyl)ethan-1-one 1 (100 mg, 0.67 mmol, 1 eq) in EtOH (2 mL) were added picolinaldehyde 2 (356.63 mg, 3.33 mmol, 5 eq), 6 M NaOH (480 mg, 13.32 mmol, 20 eq). The reaction mixture was stirred at room temperature for 5 h. The mixture was extracted with EA and washed with H₂O and brine. The product was rotary evaporated under reduced pressure with a vacuum pump, recrystallized with methanol, and filtered. After being pulled dry with a vacuum pump, a yellow solid was obtained (26.4 mg, 16.3%). 1H NMR (400 MHz, DMSO-d₆) δ 10.18 (s, 1H), 8.66 (d, J=4.8 Hz, 1H), 7.91-7.70 (m, 4H), 7.53-7.38 (m, 2H), 6.73 (d, J=8.0 Hz, 2H), 2.43 (s, 3H).

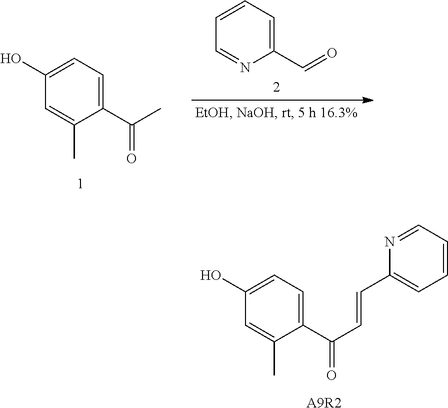

A9R3: Synthesis of Synthesis of (E)-1-(2-fluoro-4-hydroxyphenyl)-3-(pyridin-2-yl)prop-2-en-1-one. To a solution of 1-(2-fluoro-4-hydroxyphenyl)ethan-1-one 1 (100 mg, 0.65 mmol) in EtOH (2 mL), picolinaldehyde 2 (70 mg, 0.65 mmol) and NaOH (260 mg, 6.5 mmol in 0.5 mL H2O) were added. The reaction was stirred at RT for 2 h. The mixture was diluted with water and neutralized with 1 M aqueous HCl solution (pH adjusted to 7). The precipitate was filtered and collected as the crude product. The crude product was purified by methanol recrystallization to give the title compound (21 mg, Y=13%) as a yellow solid. LCMS (+ESI): m/z, 244 [M+H]+, RT=2.56 min. HPLC: 95%, RT-3.29 min. 1H NMR (400 MHZ, DMSO-d₆) δ 10.91 (s, 1H), 8.68 (dd, J=5.0, 1.8 Hz, 1H), 7.92-7.88 (m, 1H), 7.86 (t, J=2.7 Hz, 1H), 7.81-7.75 (m, 2H), 7.64 (dd, J=15.3, 1.6 Hz, 1H), 7.43 (ddd, J=7.6, 4.7, 1.2 Hz, 1H), 6.77 (dd, J=8.6, 2.3 Hz, 1H), 6.68 (dd, J=13.5, 2.3 Hz, 1H).

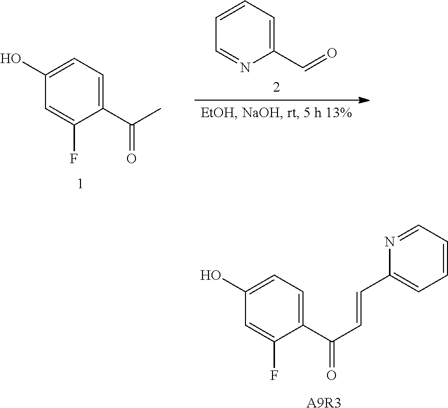

A9R4: Synthesis of 1-(2-hydroxy-4-((tetrahydro-2H-pyran-2-yl)oxy)phenyl)ethan-1-one. A mixture of 1-(2,4-dihydroxyphenyl)ethan-1-one 1 (1 g, 6 mmol) and PPTS (33 mg, 0.13 mmol) in DCM, 3,4-dihydro-2H-pyran (1.6 g, 20.0 mmol) was added. The reaction mixture was stirred at R.T. for 1 h. TLC showed that the reaction was completed. The solution was extracted with DCM for 3 times, the organic layer was washed with H₂O for 3 times, brine, dried over Na₂SO₄ and concentrated in vacuo. The crude product was purified by silica gel chromatography (PE:EA=10:1) to give 1-(2-hydroxy-4-((tetrahydro-2H-pyran-2-yl)oxy)phenyl)ethan-1-one 2 (1.3 g, 75%) as an off-white solid.

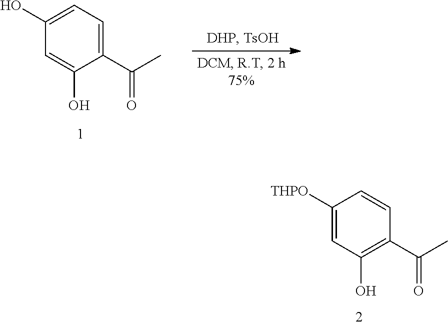

Synthesis of (E)-1-(2,4-dihydroxyphenyl)-3-(pyridin-2-yl)prop-2-en-1-one. A mixture of 1-(2-hydroxy-4-((tetrahydro-2H-pyran-2-yl)oxy)phenyl)ethan-1-one 2 (0.5 g, 2 mmol) and picolinaldehyde 3 (0.23 g, 2 mmol) in EtOH. NaOH (0.34 g, 8 mmol) was added. The reaction mixture was stirred at rt for 24 h. TLC shows 50% SM remaining. The solution was added 4N HCl adjust pH˜4, then the mixture was stirred at R.T. for 1 h, The orange solid was collected by filtration, washed with EtOH for 3 times, and dried to provide (E)-1-(2,4-dihydroxyphenyl)-3-(pyridin-2-yl)prop-2-en-1-one C9529-4 (60 mg, 11%) as a yellow solid. 1H NMR (400 MHZ, DMSO-d₆) δ 13.05 (s, 1H), 10.97 (s, 1H), 8.80 (d, J=5.0 Hz, 1H), 8.40 (d, J=15.4 Hz, 1H), 8.22 (d, J=6.2 Hz, 2H), 8.13 (d, J=9.0 Hz, 1H), 7.82 (d, J=15.4 Hz, 1H), 7.73-7.64 (m, 1H), 6.47 (dd, J=8.9, 2.3 Hz, 1H), 6.35 (d, J=2.3 Hz, 1H).

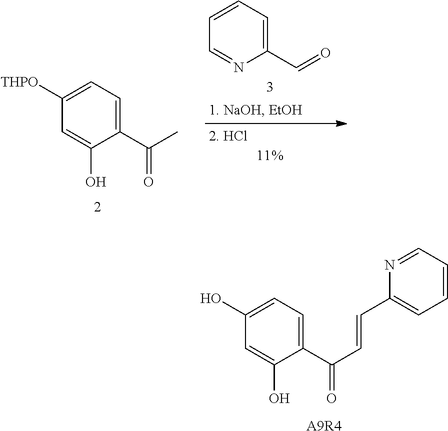

### Example 8

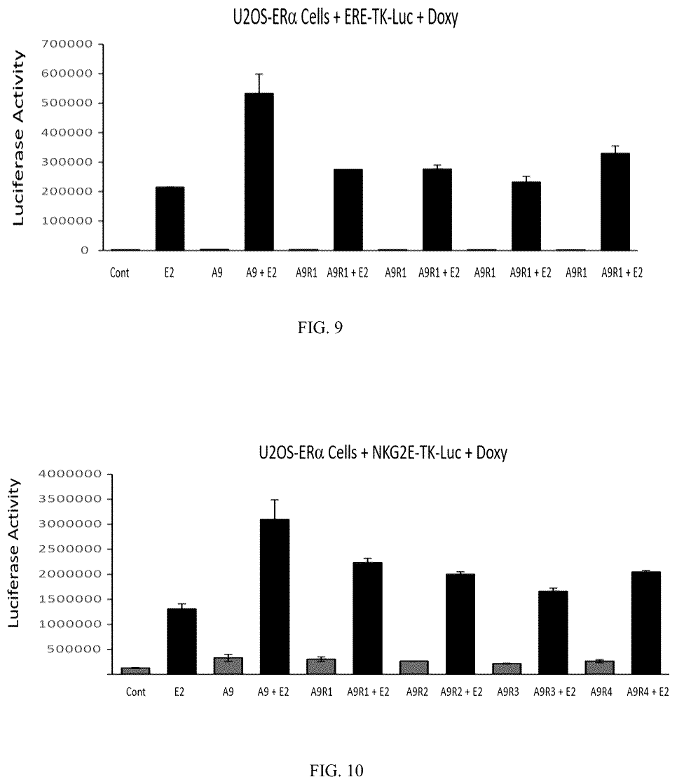

Luciferase Assay of A9 analogs. In order to better understand the structure-activity relationship of analogs of A9, the analogs A9R1, A9R2, A9R3, and A9R4 were prepared. See structures on FIG. 8. ERα-transfected doxycycline-dependent U2OS cells transfected with either ER-TK-Luc or NKG2E-TK-Luc were incubated with E2, A9, A9R1, A9R2, A9R3, or A9R4, or E2 in combination with A9, A9R1, A9R2, A9R3, or A9R4. Luciferase activity results are shown in FIG. 9 (ER-TK-Luc) and FIG. 10 (NKG2E-TK-Luc). Surprisingly, A9 demonstrated the greatest synergistic effect with E2.

### Example 8

### MCF-7 Cells, 6 Days After Treatment

The growth-promoting properties of A9 and the analogs depicted in FIG. 8 were studied in MCF-7 breast cancer cells, alone and in combination with E2. MCF-7 cells were incubated for 24 hr in the presence of medium (control), 1 nM E2, or 5 μM of each of the compounds depicted in FIG. 8 (A9 and four analogs, A9R1, A9R2, A9R3, A9R4). Viable cell counts were determined by flow cytometry.

MCF-7 cells were plated at a density of 50,000 cells per well in 6-well tissue culture plates in DMEM/F12 supplemented with 5% stripped FBS. The next day the cells were treated with vehicle or E2 in the absence and presence of A9 or one of the four analogs in FIG. 8 for 7 days. The cells were then detached with trypsin, neutralized with media containing 5% FBS, and resuspended. Appropriate amounts of cell suspension were placed in ISOTON II diluent (Thermo Fisher Scientific, Waltham, MA, USA) and the cell numbers were then measured using a Coulter Counter (Beckman, Brea, CA, USA)

Flow cytometry was performed based on a previously described method (Pan et al. 2016). Briefly, the cells were plated at a density of 500,000 cells per well in 6-well tissue culture dishes in DMEM/F-12 supplemented with 5% stripped FBS for 48 h. The cultured medium was then replaced by serum-free DMEM/F12 for 24 h. The cells were then treated with vehicle, E2 without or with the 2′,3′,4′-THC or one of the six analogs in FIG. 4 for 24 h. The culture medium was then aspirated, and the cells were washed with PBS, detached with trypsin and collected by centrifugation at 1700 rpm for 5 min. The cell pellets were washed with ice cold PBS followed by centrifugation at 1700 rpm for 10 min at room temperature. The cell pellets were resuspended in 500 μL PBS containing 50 μg/mL propidium iodide, 0.1% of triton X-100, 0.1% of sodium citrate, and 10 μg/mL of RNase. The cell suspensions were then analyzed with a BD LSR II Flow Cytometer (BD Biosciences, San Jose, CA, USA) in the Flow Cytometry facility at University of California, Berkeley and the percentage of cells in cell cycle phases were determined by using FlowJo 7.6.5 (FlowJo, LLC, Ashland, OR, USA).

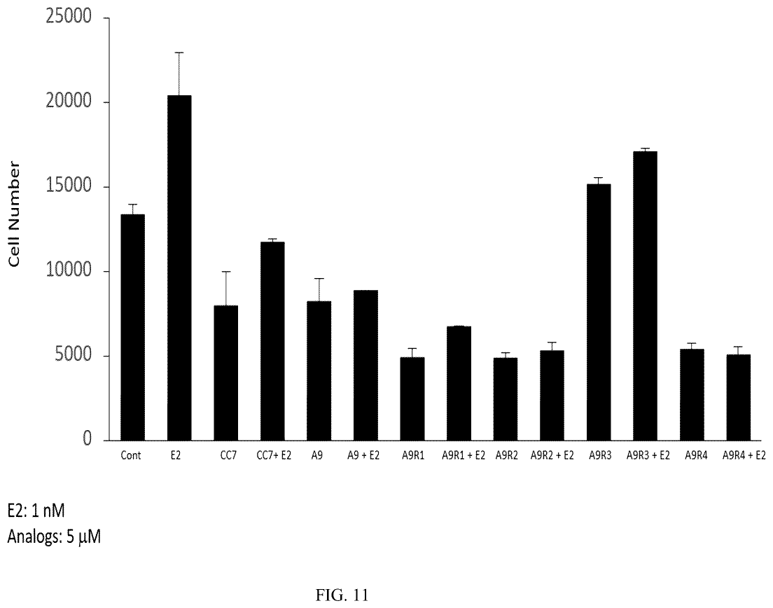

As can be seen in FIG. 11, E2 alone stimulated breast cancer cell proliferation. In contrast, the viable cell counts for CC7, A9 and each of the four analogs of A9 were well below the cell counts for E2 alone. The cell counts for CC7+E2, A9+E2, A9R1+E2, A9R2+E2 and A9R4+E2 were well below the control, which was below the cell count for E2. These results show that CC7, A9 and the four analogs of A9 do not stimulate MCF-7 breast cancer cell proliferation like E2.

Statistical analysis: All data are presented as the mean±SE or SD from at least biological triplicates. The statistical significance of the difference between two groups was assessed by the Student's t-test. For the data sets consisting of more than two groups, the statistical significance of differences among various groups (treatments) were analyzed by one-way analysis of variance (one-way ANOVA) tests or two-way ANOVA as specified in figure legends. All ANOVA tests were followed by Tukey's or Sidak's multiple comparisons post hoc tests to analyze the significance of differences between any two different treatment groups or control, as indicated in the figure legend. Statistical analysis and graph plotting were performed using GraphPad Prism version 6 (GraphPad Software Inc., San Diego, CA, USA). The statistical significance for the numbers of asterisks in the figures are *p<0.05; **p<0.01, ***p<0.001, and ****p<0.0001.

While a number of embodiments of pharmaceutical compositions and estrogenic methods are described herein, one skilled in the art understand that the examples may be altered to provide other embodiments that utilize an NRRP compound as described herein compositions and methods described herein. Therefore, it will be appreciated that the scope of this invention is to be defined by the appended claims rather than by the specific embodiments that have been represented by way of example.

1. (canceled)

2. (canceled)

3. A nuclear receptor reprogramming compound, wherein the nuclear receptor reprogramming compound is not an agonist, an antagonist, or a mixed agonist/antagonist of estrogen receptors, and wherein the compound modulates estradiol (E2) activity only when E2 is present at a physiological level, and wherein the compound has a structure of Formula I, or a pharmaceutically acceptable salt thereof, wherein the compound of Formula I is: 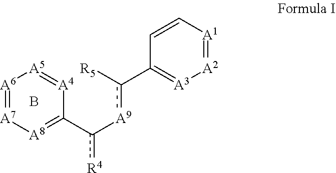
wherein,
each [symbol] is a single or double bond;
A¹, A², and A³ are independently CR¹ or N,
A⁴ is CR⁶ or N;
A⁵, A⁶, and A⁷ are independently CR² or N,
A⁸ is CR⁷ or N,
A⁹ is CR³, CHR³, N, or NR³,
each R¹ is independently H, halo, C₁-C₄ alkyl, OH, NH₂, or O—C₁-C₄ alkyl;
each R² is independently H or OH,
R³ is H or C₁-C₄ alkyl,
R⁴ is O or OH, or together with R⁷ forms a pyrido or pyrimidino ring to form a quinoline, isoquinoline or quinazoline bicyclic ring system;
R⁵ is H or, together with R⁶, forms an —O— bridge to form a benzopyran bicyclic ring system
R⁶ is H or HO, or together with R⁵ forms an —O— bridge to form a benzopyran bicyclic ring; and
R⁷ is H, OH, or together with R⁴ forms a pyrido or pyrimidine ring to form a quionline, isoquinoline, or qunazoline bicyclic ring system.

4. The compound of claim 3, wherein each R¹ is independently H, F, CH₃, OH, NH₂, or O—CH₃.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The compound of claim 3, which is: 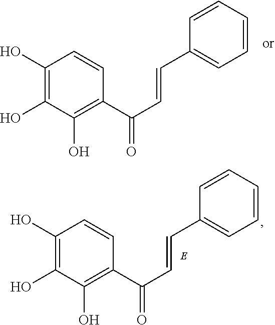 [SMILES: O=C(/C=C/c1ccccc1)c1ccc(O)c(O)c1O ; O=C(/C=C/c1ccccc1)c1ccc(O)c(O)c1O]
wherein “E” indicates the vinyl bond is entgegen.

32. (canceled)

33. The compound of claim 3, which is: 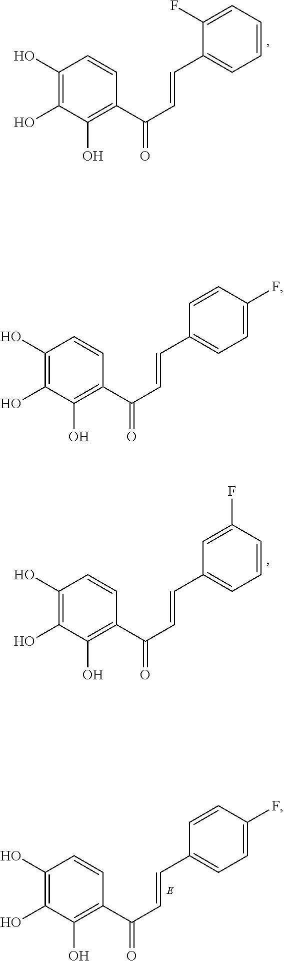 [SMILES: O=C(/C=C/c1ccccc1F)c1ccc(O)c(O)c1O ; O=C(/C=C/c1ccc(F)cc1)c1ccc(O)c(O)c1O ; O=C(/C=C/c1cccc(F)c1)c1ccc(O)c(O)c1O ; O=C(/C=C/c1ccc(F)cc1)c1ccc(O)c(O)c1O] 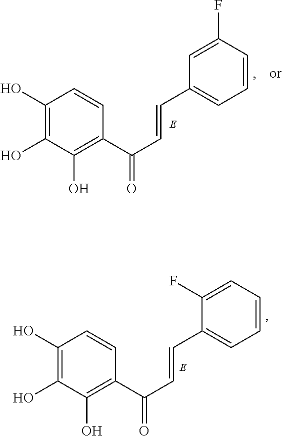 [SMILES: O=C(/C=C/c1cccc(F)c1)c1ccc(O)c(O)c1O ; O=C(/C=C/c1ccccc1F)c1ccc(O)c(O)c1O]
wherein “E” indicates the vinyl bond is entgegen.

34. (canceled)

35. The compound of claim 3, which is: 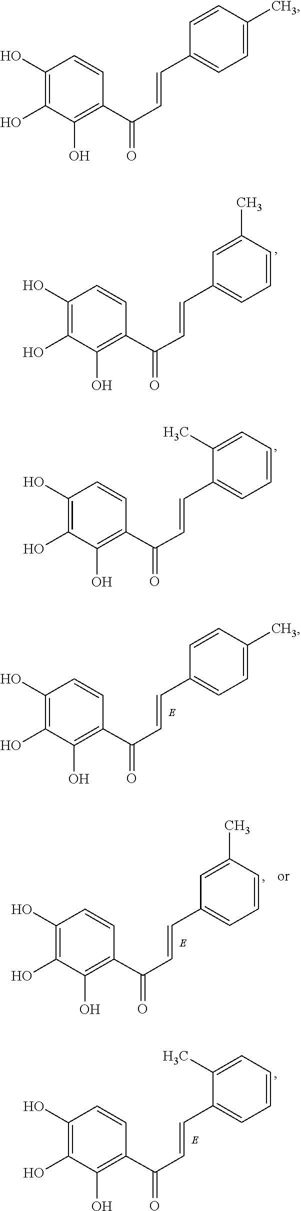 [SMILES: Cc1ccc(/C=C/C(=O)c2ccc(O)c(O)c2O)cc1 ; Cc1cccc(/C=C/C(=O)c2ccc(O)c(O)c2O)c1 ; Cc1ccccc1/C=C/C(=O)c1ccc(O)c(O)c1O ; Cc1ccc(/C=C/C(=O)c2ccc(O)c(O)c2O)cc1 ; Cc1cccc(/C=C/C(=O)c2ccc(O)c(O)c2O)c1 ; Cc1ccccc1/C=C/C(=O)c1ccc(O)c(O)c1O]
wherein “E” indicates the vinyl bond is entgegen.

36. (canceled)

37. The compound of claim 3, which is: 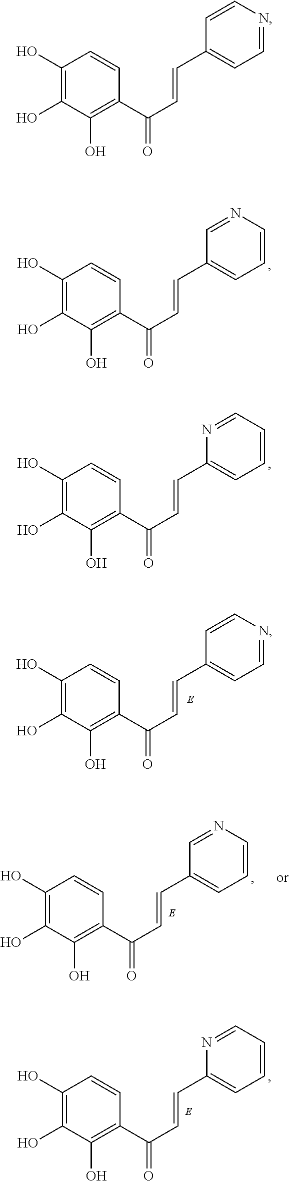 [SMILES: O=C(/C=C/c1ccncc1)c1ccc(O)c(O)c1O ; O=C(/C=C/c1cccnc1)c1ccc(O)c(O)c1O ; O=C(/C=C/c1ccccn1)c1ccc(O)c(O)c1O ; O=C(/C=C/c1ccncc1)c1ccc(O)c(O)c1O ; O=C(/C=C/c1cccnc1)c1ccc(O)c(O)c1O ; O=C(/C=C/c1ccccn1)c1ccc(O)c(O)c1O]
wherein “E” indicates the vinyl bond is entgegen.

38. (canceled)

39. The compound of claim 3, which is: 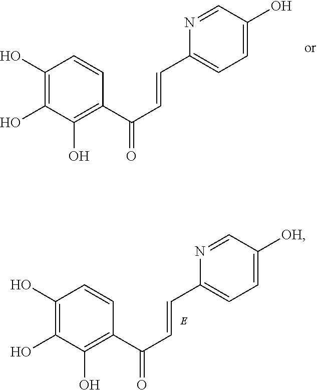 [SMILES: O=C(/C=C/c1ccc(O)cn1)c1ccc(O)c(O)c1O ; O=C(/C=C/c1ccc(O)cn1)c1ccc(O)c(O)c1O]
wherein “E” indicates the vinyl bond is entgegen.

40. (canceled)

41. The compound of claim 3, which is: 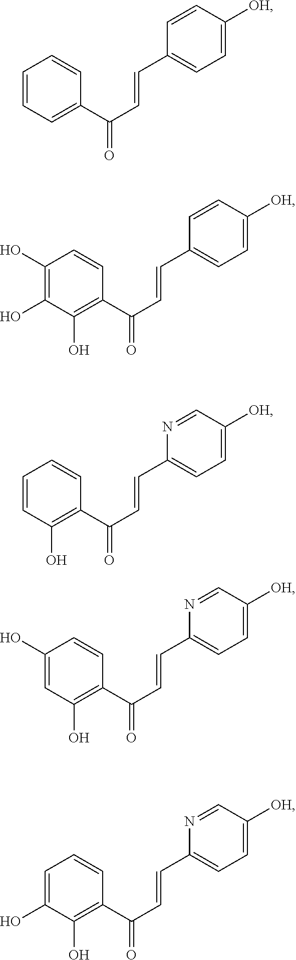 [SMILES: O=C(/C=C/c1ccc(O)cc1)c1ccccc1 ; O=C(/C=C/c1ccc(O)cc1)c1ccc(O)c(O)c1O ; O=C(/C=C/c1ccc(O)cn1)c1ccccc1O ; O=C(/C=C/c1ccc(O)cn1)c1ccc(O)cc1O ; O=C(/C=C/c1ccc(O)cn1)c1cccc(O)c1O] 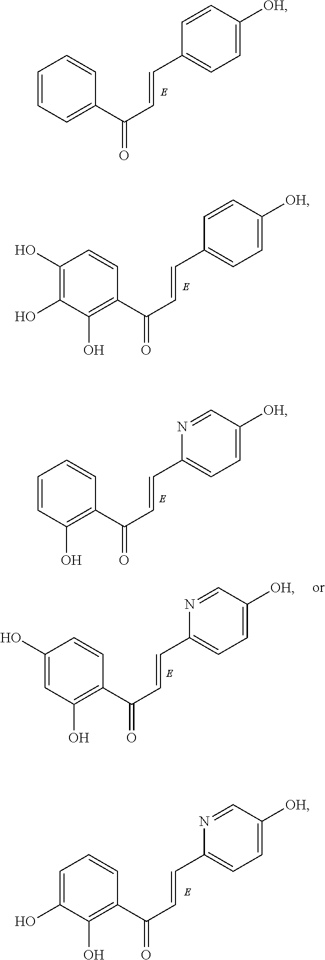 [SMILES: O=C(/C=C/c1ccc(O)cc1)c1ccccc1 ; O=C(/C=C/c1ccc(O)cc1)c1ccc(O)c(O)c1O ; O=C(/C=C/c1ccc(O)cn1)c1ccccc1O ; O=C(/C=C/c1ccc(O)cn1)c1ccc(O)cc1O ; O=C(/C=C/c1ccc(O)cn1)c1cccc(O)c1O]
wherein “E” indicates the vinyl bond is entgegen.

42. (canceled)

43. The compound of claim 3, which is: 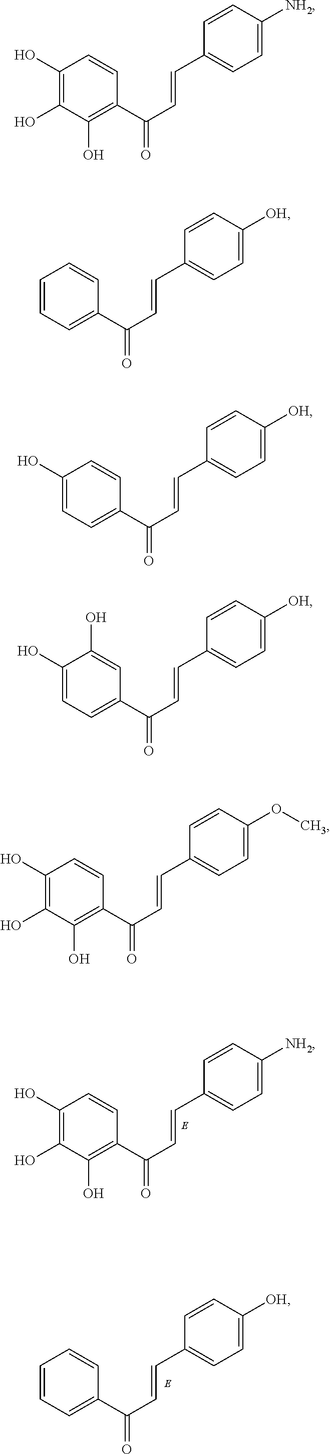 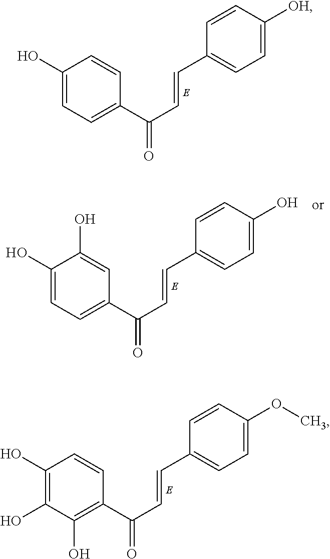 [SMILES: O=C(/C=C/c1ccc(O)cc1)c1ccc(O)cc1 ; O=C(/C=C/c1ccc(O)cc1)c1ccc(O)c(O)c1 ; COc1ccc(/C=C/C(=O)c2ccc(O)c(O)c2O)cc1]
wherein “E” indicates the vinyl bond is entgegen.

44. (canceled).

45. The compound of claim 3, which is: 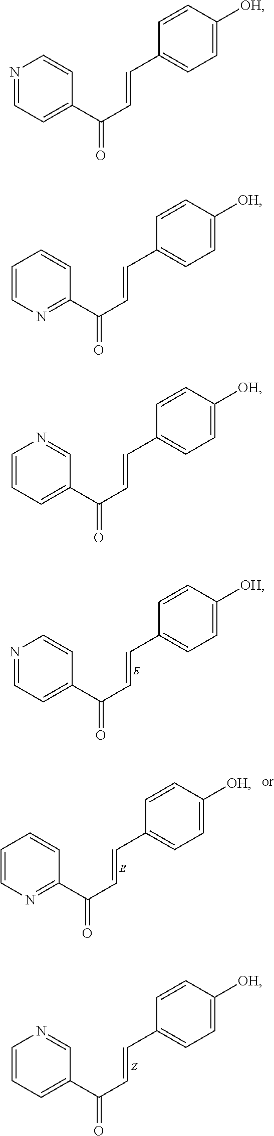
wherein “E” indicates the vinyl bond is entgegen.

46. (canceled)

47. The compound of claim 3, which is: 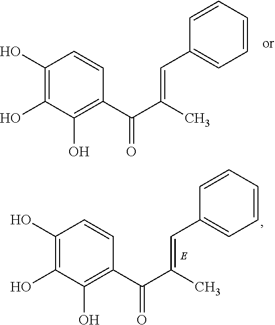 [SMILES: C/C(=C\c1ccccc1)C(=O)c1ccc(O)c(O)c1O ; C/C(=C\c1ccccc1)C(=O)c1ccc(O)c(O)c1O]
wherein “E” indicates the vinyl bond is entgegen.

48. The compound of claim 3, which is:

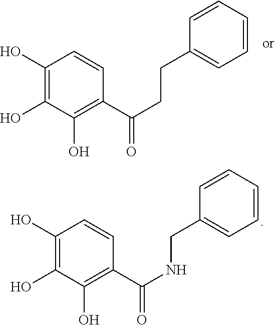 [SMILES: O=C(CCc1ccccc1)c1ccc(O)c(O)c1O ; O=C(NCc1ccccc1)c1ccc(O)c(O)c1O]

49. The compound of claim 3, which is: 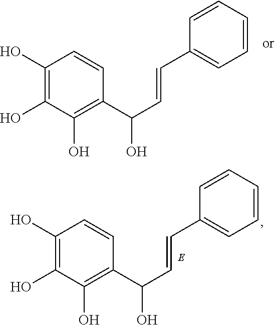 [SMILES: Oc1ccc(C(O)/C=C/c2ccccc2)c(O)c1O ; Oc1ccc(C(O)/C=C/c2ccccc2)c(O)c1O]
wherein E indicates the vinyl bond is entgegen.

50. The compound of claim 3, which is:

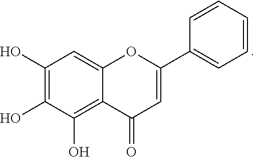 [SMILES: O=c1cc(-c2ccccc2)oc2cc(O)c(O)c(O)c12]

51. The compound of claim 3, which is:

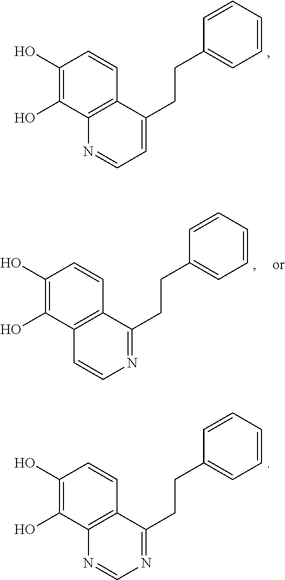 [SMILES: Oc1ccc2c(CCc3ccccc3)ccnc2c1O ; Oc1ccc2c(CCc3ccccc3)nccc2c1O ; Oc1ccc2c(CCc3ccccc3)ncnc2c1O]

52. The compound of claim 3, or a pharmaceutically acceptable salt thereof, wherein the compound has one of the following structures:

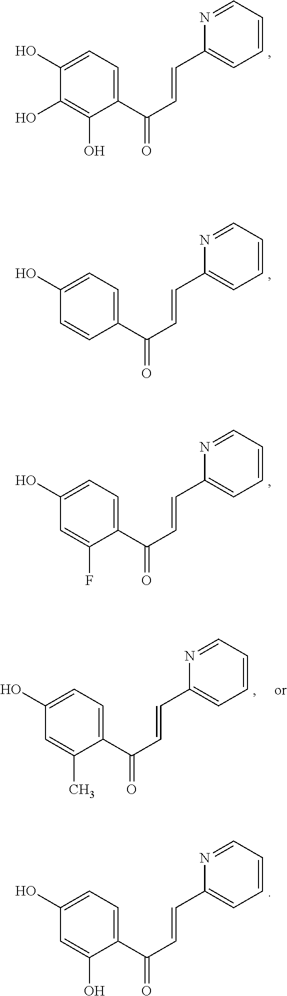 [SMILES: O=C(/C=C/c1ccccn1)c1ccc(O)c(O)c1O ; O=C(/C=C/c1ccccn1)c1ccc(O)cc1 ; O=C(/C=C/c1ccccn1)c1ccc(O)cc1F ; Cc1cc(O)ccc1C(=O)/C=C/c1ccccn1 ; O=C(/C=C/c1ccccn1)c1ccc(O)cc1O]

53. (canceled)

54. A pharmaceutical composition comprising a compound of claim 3, or a pharmaceutically acceptable salt thereof, and at least one pharmaceutically acceptable excipient.

55. The pharmaceutical composition of claim 54, further comprising a member of the group consisting of estradiol (E2), one or more estrogen receptor agonists, one or more estrogen receptor antagonists, one or more mixed estrogen receptor agonist/antagonists, one or more selective estrogen receptor modulators (SERMs), one or more progestogens, one or more glucocorticoids, and one or more androgens.

56. (canceled)

57. (canceled)

58. (canceled)

59. (canceled)

60. (canceled)

61. (canceled)

62. A method of treating a condition or disease state in a patient in need of such treatment, comprising administering to the patient an effective amount of a compound of claim 3, wherein the condition or disease state is a development condition, a menstruation condition, a fertility condition, a gynecological disease, an autoimmune disorder, a menopause disorder, an aging disorder, or cancer.

63. (canceled)

64. The method of claim 62, wherein the condition or disease state is Turner Syndrome, Kallmann Syndrome, congenital primary amenorrhea, a childhood neuropsychiatric disorder, dysmenorrhea, amenorrhea, menorrhagia, estrogen-induced deep vein thrombosis, pulmonary embolism, conception, fetal implantation, spontaneous abortion, preterm labor, endometriosis, polycystic ovarian syndrome, rheumatoid arthritis, scleroderma, Sjogren's syndrome, Hashimoto's thyroiditis, multiple sclerosis, irritable bowel syndrome, ulcerative colitis, Crohn's disease, vasomotor symptoms of menopause and perimenopause, insomnia, nighttime wakening, mood swings, vulvovaginal atrophy, vaginal dryness, dyspareunia, menopausal weight gain and obesity, osteoporosis, type-2 diabetes, estrogen-induced deep vein thrombosis and pulmonary embolism, Alzheimer's disease, early dementia, breast cancer, uterine cancer, ovarian cancer, prostate cancer, and non-small cell lung cancer.
